(19) United States
(12) Patent Application Publication
(10) Pub. No.: US 2006/0263802 A1
(43) Pub. Date: Nov. 23, 2006

(54) Antigenic peptides of rabies virus and uses thereof

(75) Inventors: Alexander Berthold Hendrik Bakker, Hillegom (NL); Willem Egbert Marissen, Woerden (NL); Jaap Goudsmit, Amsterdam (NL)

Correspondence Address: TRASK BRITT, P.O. BOX 2550, SALT LAKE CITY, UT, 84110, US

(73) Assignee: Crucell Holland B.V., Leiden (NL)

(21) Appl. No.: 11/361,661

(22) Filed: Feb. 23, 2006

Related U.S. Application Data
(63) Continuation of application No. PCT/EP04/52043, filed on Sep. 3, 2004.

(30) Foreign Application Priority Data
Sep. 4, 2003 (WO) PCT/EP03/50396
Jun. 28, 2004 (WO) PCT/EP04/51274

(51) Int. Cl. C12Q 1/68; C12N 5/06; C12N 5/16

(52) U.S. Cl. 435/6; 435/345

(57) ABSTRACT

The present invention pertains to antigenic peptides of rabies virus and their use in the detection, prevention and/or treatment of conditions resulting from rabies virus.

### CROSS-REFERENCE TO RELATED APPLICATIONS

This application is a continuation of PCT International Patent Application No. PCT/EP2004/052043, filed on Sep. 3, 2004, designating the United States of America, and published, in English, as PCT International Publication No. WO 2005/023849 A2 on Mar. 17, 2005, which claims priority to PCT International Patent Application No. PCT/EP03/50396, filed on Sep. 4, 2003, and PCT/EP04/051274, filed on Jun. 28, 2004, the contents of the entirety of each of which are hereby incorporated herein by this reference.

### STATEMENT ACCORDING TO 37 C.F.R. § 1.52(e)(5)-SEQUENCE LISTING SUBMITTED ON COMPACT DISC

Pursuant to 37 C.F.R. § 1.52(e)(1)(ii), a compact disc containing an electronic version of the Sequence Listing has been submitted concomitant with this application, the contents of which are hereby incorporated by reference. A second compact disc is submitted and is an identical copy of the first compact disc. The discs are labelled “Copy 1” and “Copy 2,” respectively, and each disc contains one file entitled “2578-7684US seq list.txt” which is 168 KB and created on Feb. 23, 2006.

### TECHNICAL FIELD

In general, embodiments of the invention relate to biotechnology. More particularly, embodiments of the present invention relate to medicine. In particular, the invention relates to antigenic peptides of rabies virus and uses thereof.

### BACKGROUND

Rabies is a viral infection with nearly worldwide distribution that affects principally wild and domestic animals but also involves humans, resulting in a devastating, almost invariable fatal encephalitis. Annually, more than 70,000 human fatalities are estimated, and millions of others require post-exposure treatment.

The rabies virus is a bullet-shaped, enveloped, single-stranded RNA virus classified in the rhabdovirus family and Lyssavirus genus. The genome of rabies virus codes for five viral proteins: RNA-dependent RNA polymerase (L); a nucleoprotein (N); a phosphorylated protein (P); a matrix protein (M) located on the inner side of the viral protein envelope; and an external surface glycoprotein (G).

Rabies can be treated or prevented by both passive and active immunizations. Currently, a number of anti-rabies vaccines based on inactivated or attenuated virus exist (U.S. Pat. Nos. 4,347,239, 4,040,904, and 4,752,474). However, there are risks associated with these vaccines. The vaccines that contain inactivated or attenuated virus occasionally produce neurologic or central nervous system disorders in those vaccinated. Further, there is a risk that all of the virus in a lot of supposedly inactivated-virus vaccine will not be killed, or that some of the virus in a lot of attenuated-virus vaccine will revert to a virulent state, and that rabies might be caused in an individual mammal by vaccination with a dose which happens to contain live, virulent virus. Moreover, the vaccines are produced in tissue culture and are, therefore, expensive to produce. Vaccines based on coat glycoprotein isolated from the virus entail many of the risks associated with inactivated- or attentuated-virus vaccines, because obtaining coat glycoprotein involves working with live virus.

The above disadvantages are not found in synthetic vaccines. The key to developing such a vaccine is identifying antigenic peptides on the glycoprotein of rabies virus that have sequences of amino acids that are continuous, i.e., the peptides are uninterrupted fragments of the primary structure of the protein on which the peptides occur. Such antigenic peptides have been described (see Luo et al. 1997 and Dietzschold et al. 1990), but their effectiveness, efficacy and broadness is limited and has to be improved. Therefore, there remains a need for a vaccine for rabies virus that is of potency and broadness superior to the described vaccines.

It has now been found that there are other antigenic peptides beyond those discovered. The sequence of these peptides is highly conserved among the various rabies virus strains. Thus, a vaccine with a synthetic peptide with such a sequence will not be limited by antigenic variability and will offer the potential that they can be used as vaccinating agents to generate antibodies useful for prevention and/or treatment of a wide range of rabies viruses.

### SUMMARY OF THE INVENTION

The present invention generally relates to antigenic peptides of rabies virus. Furthermore, various embodiments of the invention provide fusion proteins comprising these peptides. Further embodiments comprise methods for prevention and/or treatment of a condition resulting from a rabies virus.

### DESCRIPTION OF THE FIGURES

FIG. 1: PEPSCAN-analysis of the extracellular domain of the surface glycoprotein G from rabies virus strain ERA. Binding of the human monoclonal antibodies CRJA, CRJB and CR57 is tested in a PEPSCAN-based enzyme-linked immunoassay and quantified with a CCD-camera and an image processing system. On the Y-axis, the OD values are shown. The left peak corresponds with the sequence YDRSLHSRVFPSGKC (SEQ ID NO:2) and the high peak(s) corresponds with the sequence SLKGACKLKLCGVLGLRLMDGTW (SEQ ID NO:56).

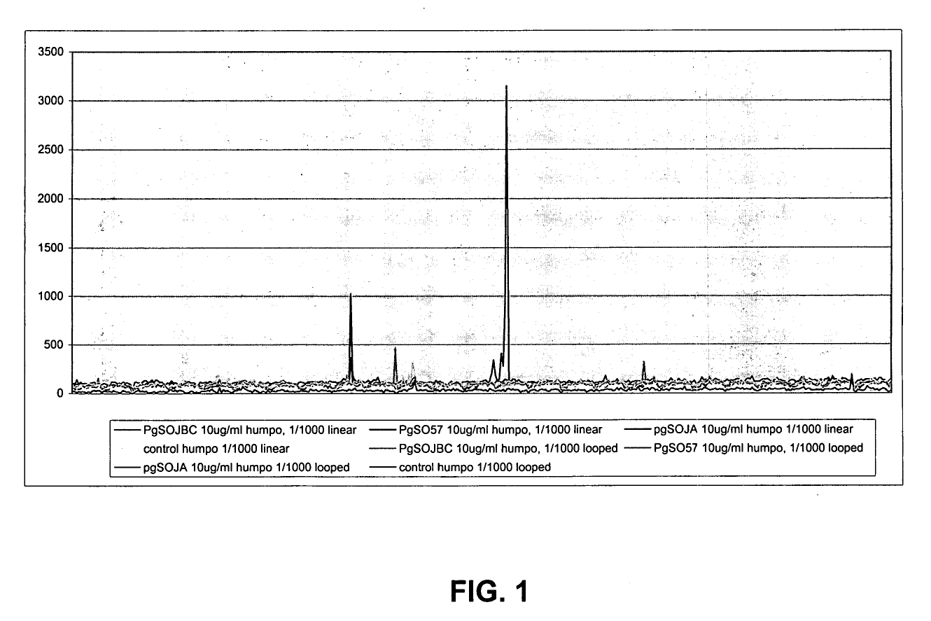

FIG. 2: Amino acid sequence (SEQ ID NO:19) of the surface glycoprotein G from rabies virus strain ERA. The extracellular domain consists of amino acids 20-458. The signal peptide sequence consists of amino acids 1 -19.

FIG. 3: Comparison of epitope defined by amino acids 164-178 among several genotype 1 rabies virus strains. Amino acids that are not identical to the ERA sequence are shown in bold. The SEQ ID NOs of the sequences shown in FIG. 3 are, from top to bottom, SEQ ID NO:2, SEQ ID NO:44, SEQ ID NO:44, SEQ ID NO:45, SEQ ID NO:2, SEQ ID NO:46, SEQ ID NO:46, SEQ ID NO:46, SEQ ID NO:46, SEQ ID NO:47, SEQ ID NO:47, SEQ ID NO:48, SEQ ID NO:46, SEQ ID NO:49, SEQ ID NO:46, SEQ ID NO:46, SEQ ID NO:46, SEQ ID NO:47, SEQ ID NO:46, SEQ ID NO:46, SEQ ID NO:46, SEQ ID NO:46, SEQ ID NO:46 and SEQ ID NO:46.

FIG. 4: Comparison of epitope defined by amino acids 164-178 among Lyssavirus genotypes 1-7. Amino acids that are not identical to the ERA sequence are shown in bold. The SEQ ID NOs of the sequences shown in FIG. 4 are, from top to bottom, SEQ ID NO:2, SEQ ID NO:50, SEQ ID NO:51, SEQ ID NO:52, SEQ ID NO:53, SEQ ID NO:54 and SEQ ID NO:55.

FIG. 5: Comparison of epitope defined by amino acids 237-259 among several genotype 1 rabies virus strains. Amino acids that are not identical to the ERA sequence are shown in bold. The SEQ ID NOs of the sequences shown in FIG. 5 are, from top to bottom, SEQ ID NO:56, SEQ ID NO:56, SEQ ID NO:56, SEQ ID NO:56, SEQ ID NO:56, SEQ ID NO:57, SEQ ID NO:57, SEQ ID NO:57, SEQ ID NO:57, SEQ ID NO:56, SEQ ID NO:56, SEQ ID NO:56, SEQ ID NO:58, SEQ ID NO:56, SEQ ID NO:56, SEQ ID NO:56, SEQ ID NO:56, SEQ ID NO:56, SEQ ID NO:56, SEQ ID NO:56, SEQ ID NO:56, SEQ ID NO:56, SEQ ID NO:56 and SEQ ID NO:59.

FIG. 6: Comparison of epitope defined by amino acids 237-259 among Lyssavirus genotypes 1-7. Amino acids that are not identical to the ERA sequence are shown in bold. The SEQ ID NOs of the sequences shown in FIG. 6 are, from top to bottom, SEQ ID NO:56, SEQ ID NO:60, SEQ ID NO:61, SEQ ID NO:62, SEQ ID NO:63, SEQ ID NO:64 and SEQ ID NO:65.

FIG. 7 shows comparison of amino acid sequences of the rabies virus strain CVS-11 and E57 escape viruses. Virus-infected cells were harvested two days post-infection and total RNA was isolated. cDNA was generated and used for DNA sequencing. Regions containing mutations are shown and the mutations are indicated in bold. FIG. 7A shows the comparison of the nucleotide sequences. Numbers above amino acids indicate amino acid numbers from rabies virus glycoprotein including signal peptide. FIG. 7B shows the comparison of amino acid sequences. Schematic drawing of rabies virus glycoprotein is shown on top. The black box indicates the signal peptide, while the gray box indicates the transmembrane domain. The sequences in FIG. 7 are also represented by SEQ ID NOs:66-77.

FIG. 8 shows comparison of amino acid sequences of the rabies virus strain CVS-11 and EJB escape viruses. Virus-infected cells were harvested two days post-infection and total RNA was isolated. cDNA was generated and used for DNA sequencing. Regions containing mutations are shown and the mutations are indicated in bold. FIG. 8A shows the comparison of the nucleotide sequences. Numbers above amino acids indicate amino acid numbers from rabies virus glycoprotein including the signal peptide. FIG. 8B shows the comparison of amino acid sequences. Schematic drawing of rabies virus glycoprotein is shown on top. The black box indicates the signal peptide, while the gray box indicates the transmembrane domain. The sequences in FIG. 8 are also represented by SEQ ID NOs:78-87 (wherein SEQ ID NO:85 is identical to SEQ ID NO:74 shown in FIG. 7).

FIG. 9: PEPSCAN-analysis of 12-, 10-, and 8-mer peptides spanning the region SLKGACKLKLCGVLGLRLMDGTW (from the ERA rabies strain; SEQ ID NO:56) or SLKGACRLKLCGVLGLRLMDGTW (from the CVS-11 rabies strain; SEQ ID NO:74). The two sequences differ in that a lysine is substituted for an arginine. Binding of the human monoclonal antibody CR57 is tested in a PEPSCAN-based enzyme-linked immuno assay and quantified with a CCD-camera and an image processing system. On the Y-axis, the OD values and on the X-axis, the peptides of the region SLKGACKLKLCGVLGLRLMDGTW (SEQ ID NO:56) are shown. The left (dark) bars are the data of the peptides of SLKGACKLKLCGVLGLRLMDGTW (SEQ ID NO:56) and the right (light) bars, the data of the peptides of SLKGACRLKLCGVLGLRLMDGTW (SEQ ID NO:74).

FIG. 10: Alanine replacement scanning analysis in combination with PEPSCAN-analysis of an 8-mer peptide spanning the region LKLCGVLG (SEQ ID NO:98). Binding of the human monoclonal antibody CR57 is tested in a PEPSCAN-based enzyme-linked immunoassay and quantified with a CCD-camera and an image processing system. On the Y-axis, the OD values and on the X-axis, the different peptides are shown. FIG. 10 additionally shows the binding of CR57 to the peptides LELCGVLG (SEQ ID NO: 100, LNLCGVLG (SEQ ID NO:101) and LKLCEVLG (SEQ ID NO:102) harboring the mutations observed in the epitope in E57 escape viruses.

### DETAILED DESCRIPTION OF THE INVENTION

In a first aspect, the invention provides antigenic peptides of rabies virus. The antigenic peptides of the invention comprise an amino acid sequence KX₁CGVX₂ (SEQ ID NO: 104), wherein X₁ and X₂ may be any amino acid residue and wherein X₁ and X₂ may be the same or different from one another.

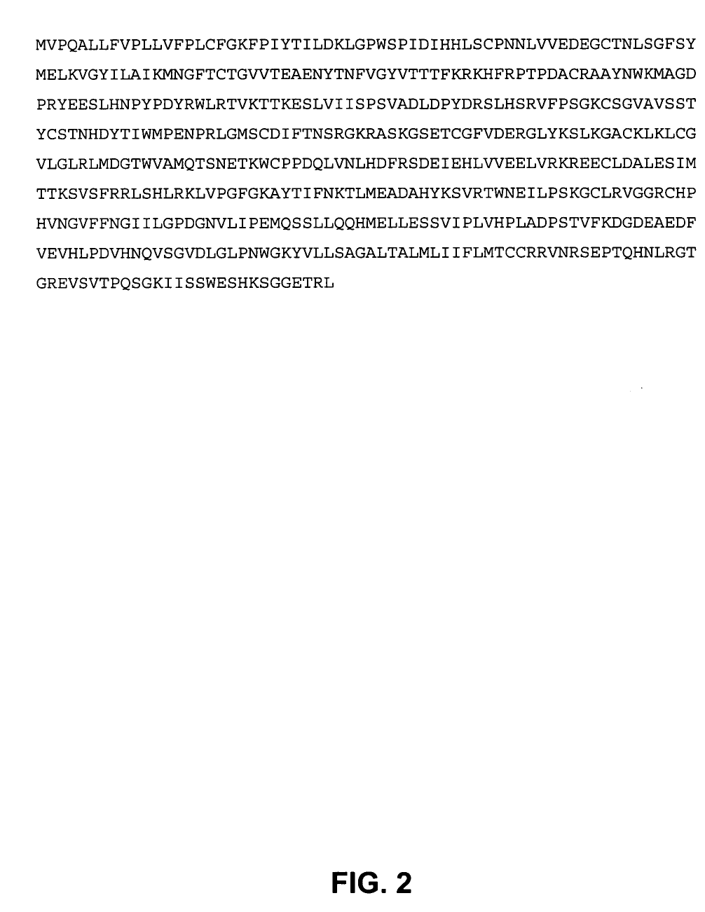

In the present invention, binding of three monoclonal antibodies called CRJA, CRJB and CR57 to a series of overlapping 15-mer peptides, which were either in linear form or in looped/cyclic form, of the glycoprotein G from rabies virus, in particular, the extracellular part of the glycoprotein G of rabies virus strain ERA, was analyzed by means of PEPSCAN analysis (see, inter alia WO 84/03564, WO 93/09872, Slootstra et al. 1996). The glycoprotein of rabies virus strain ERA (the protein-id of the glycoprotein of rabies virus strain ERA in the EMBL-database is AAA47204.1; the gene can be found in the database under J02293; for the amino acid sequence of the glycoprotein of rabies virus strain ERA, see also FIG. 2 and SEQ ID NO:19) is highly homologous to the glycoprotein G of other rabies virus strains. Particularly, the extracellular domain of glycoprotein G of the rabies virus strain ERA appears to have a high homology with the extracellular domain of other rabies virus strains. In general, rabies virus glycoprotein (G) is composed of a cytoplasmic domain, a transmembrane domain, and an extracellular domain. The glycoprotein is a trimer, with the extracellular domains exposed at the virus surface.

The antigenic peptides of the invention are derived from a rabies virus glycoprotein, preferably the extracellular domain thereof. Preferably, the peptides are common to a plurality of differing rabies virus strains and are capable of eliciting rabies virus-neutralizing antibodies, preferably antibodies capable of neutralizing different rabies virus strains. In a preferred embodiment, the peptides are recognized by the neutralizing anti-rabies virus antibody called CR57.

The antigenic peptides found in the present invention may not only be used for detection, prevention and/or treatment of a condition resulting from the rabies virus strain ERA, but may also be useful in detecting, preventing and/or treating a condition resulting from rabies viruses in general and might even be used to prevent and/or treat a condition resulting from a virus of the Lyssavirus genus and even a virus of the rhabdovirus family.

In one embodiment, the invention provides a peptide having an amino acid sequence selected from the group consisting of GYVTTTFKRKHFRPT (SEQ ID NO:1), YDRSLHSRVFPSGKC (SEQ ID NO:2), YTIWMPENPRLGMSC (SEQ ID NO:3), IWMPENPRLGMSCDI (SEQ ID NO:4), WMPENPRLGMSCDIF (SEQ ID NO:5), SLKGACKLKLCGVLG (SEQ ID NO:6), LKGACKLKLCGVLGL (SEQ ID NO:7), KGACKLKLCGVLGLR (SEQ ID NO:8), GACKLKLCGVLGLRL (SEQ ID NO:9), ACKLKLCGVLGLRLM (SEQ ID NO:10), CKLKLCGVLGLRLMD (SEQ ID NO:11), KLKLCGVLGLRLMDG (SEQ ID NO:12), LKLCGVLGLRLMDGT (SEQ ID NO:13) and KLCGVLGLRLMDGTW (SEQ ID NO: 14), NHDYTIWMPENPRLG (SEQ ID NO: 15), DPYDRSLHSRVFPSG (SEQ ID NO:16), YCSTNHDYTIWMPEN (SEQ ID NO:17) and SFRRLSHLRKLVPGF (SEQ ID NO:18).

The peptides above are recognized by at least one of the human monoclonal antibodies called CRJB, CR57 and CRJA antibodies known to bind to rabies virus. The original generation of antibody CRJA is described in detail in WO 01/088132. The GenBank Accession No. of the light chain of CRJA is AY172961. The GenBank Accession No. of the heavy chain of CRJA is AY172959. The original generation of antibodies CRJB and CR57 is described in detail in WO 03/016501 and U.S. 2003/0157112. The GenBank Accession No. of the light chain of CRJB is AY172962. The GenBank Accession No. of the heavy chain of CRJB is AY172958. The GenBank Accession No. of the light chain of CR57 is AY172960 (the variable part of this light chain can also be found under Genbank Accession No. D84141; the sequence of D84141 contains two silent mutations in the CDR3 region). The GenBank Accession No. of the heavy chain of CR57 is AY172957.

In another embodiment, the invention encompasses a peptide having an amino acid sequence selected from the group consisting of GYVTTTFKRKHFRPT (SEQ ID NO:1), YDRSLHSRVFPSGKC (SEQ ID NO:2), YTIWMPENPRLGMSC (SEQ ID NO:3), IWMPENPRLGMSCDI (SEQ ID NO:4), WMPENPRLGMSCDIF (SEQ ID NO:5), SLKGACKLKLCGVLG (SEQ ID NO:6), LKGACKLKLCGVLGL (SEQ ID NO:7), KGACKLKLCGVLGLR (SEQ ID NO:8), GACKLKLCGVLGLRL (SEQ ID NO:9), ACKLKLCGVLGLRLM (SEQ ID NO:10), CKLKLCGVLGLRLMD (SEQ ID NO:11), KLKLCGVLGLRLMDG (SEQ ID NO:12), LKLCGVLGLRLMDGT (SEQ ID NO:13) and KLCGVLGLRLMDGTW (SEQ ID NO:14). These peptides are recognized in linear and/or looped form by the human monoclonal antibody called CR57.

Preferably, the peptide has an amino acid sequence selected from the group consisting of SLKGACKLKLCGVLG (SEQ ID NO:6), LKGACKLKLCGVLGL (SEQ ID NO:7), KGACKLKLCGVLGLR (SEQ ID NO:8), GACKLKLCGVLGLRL (SEQ ID NO:9), ACKLKLCGVLGLRLM (SEQ ID NO:10), CKLKLCGVLGLRLMD (SEQ ID NO:11), KLKLCGVLGLRLMDG (SEQ ID NO:12), LKLCGVLGLRLMDGT (SEQ ID NO:13) and KLCGVLGLRLMDGTW (SEQ ID NO:14). More preferably, the peptide has an amino acid sequence selected from the group consisting of LKLCGVLGLRLMDGT (SEQ ID NO:13) and KLCGVLGLRLMDGTW (SEQ ID NO:14). Particularly preferred is the peptide having the amino acid sequence KLCGVLGLRLMDGTW (SEQ ID NO:14).

In yet another embodiment, the peptide has an amino acid sequence selected from the group consisting of YDRSLHSRVFPSGKC (SEQ ID NO:2), NHDYTIWMPENPRLG (SEQ ID NO:15) and WMPENPRLGMSCDIF (SEQ ID NO:5). These peptides are recognized in linear and/or looped form by the human monoclonal antibody called CRJB.

In a further embodiment, the peptide has an amino acid sequence selected from the group consisting of DPYDRSLHSRVFPSG (SEQ ID NO:16), YDRSLHSRVFPSGKC (SEQ ID NO:2), YCSTNHDYTIWMPEN (SEQ ID NO:17) and SFRRLSHLRKLVPGF (SEQ ID NO:18). These peptides are recognized in linear and/or looped form by the human monoclonal antibody called CRJA.

In a specific embodiment, the peptide has the amino acid sequence shown in YDRSLHSRVFPSGKC (SEQ ID NO:2). This peptide is recognized in linear form by all three human monoclonal antibodies.

The combined observations lead us to believe that the oligopeptides identified above are good candidates to represent neutralizing epitopes of rabies virus. SLKGACKLKLCGVLGLRLMDGTW (SEQ ID NO:56) is a particularly interesting region of the glycoprotein based on its high reactivity in PEPSCAN. Linear peptides within this region clearly bound to the human monoclonal antibody called CR57. The presence of mutations in this region in escape viruses of CR57 and CRJB indicated that the region harbors a neutralizing epitope of the rabies glycoprotein. PEPSCAN analysis of 12-, 10-, and 8-mer linear peptides spanning this region harboring a neutralizing epitope of rabies virus and alanine replacement scanning analysis of the peptides revealed that the neutralizing epitope recognized comprises the core region or critical binding region KX₁CGVX₂ (SEQ ID NO:104), wherein X₁ and X₂ can be any amino acid residue and X₁ and X₂ can be the same or different from one another. The critical binding region is highly conserved within rabies viruses of genotype 1. In an embodiment of the invention, amino acid residues X1 and X2 are amino acid residues having nonpolar side chains such as e.g., glycine, alanine, valine, leucine, isoleucine, proline, phenylalanine, or methionine. In a specific embodiment, the amino acid residues X1 and X2 are both selected from leucine and alanine.

The peptides of the invention may be used to obtain further antibodies against the peptides. This way, the antigenicity of the peptides can be investigated. Methods for producing antibodies are well known to the person skilled in the art including, but not limited to, immunization of animals such as mice, rabbits, goats, and the like, or by antibody, phage or ribosome display methods.

In a further aspect of the invention, the peptides mentioned above may be coupled/linked to each other. In other words, the invention also encompasses a multimer of peptides, wherein the peptides are peptides of the invention. Peptides of the embodiments of the invention may be linked/coupled to peptides of other embodiments of the invention or the same embodiment of the invention. The peptides may be linear and/or looped/cyclic. A combination peptide obtained this way may mimic/simulate a discontinuous and/or conformational epitope that is more antigenic than the single peptides. The combination peptide may also constitute more than two peptides. The peptides of the invention can be linked directly or indirectly via, for instance, a spacer of variable length. Furthermore, the peptides can be linked covalently or non-covalently. They may also be part of a fusion protein or conjugate. In general, the peptides should be in such a form as to be capable of mimicking/simulating a discontinuous and/or conformational epitope.

Obviously, the person skilled in the art may make modifications to the peptide without departing from the scope of the invention, e.g., by systematic length variation and/or replacement of residues and/or combination with other peptides. Peptides can be synthesized by known solid phase peptide synthesis techniques. The synthesis allows for one or more amino acids not corresponding to the original peptide sequence to be added to the amino or carboxyl terminus of the peptides. Such extra amino acids are useful for coupling the peptides to each other, to another peptide, to a large carrier protein or to a solid support. Amino acids that are useful for these purposes include, inter alia, tyrosine, lysine, glutamic acid, aspartic acid, cysteine and derivatives thereof. Additional protein modification techniques may be used, e.g., NH₂-acetylation or COOH-terminal amidation, to provide additional means for coupling the peptides to another protein or peptide molecule or to a support, for example, polystyrene or polyvinyl microtiter plates, glass tubes or glass beads or particles and chromatographic supports, such as paper, cellulose and cellulose derivates, and silica. If the peptide is coupled to such a support, it may also be used for affinity purification of anti-rabies virus antibodies recognizing the peptide.

The peptides of the invention may have a varying size. They may contain at least 100, at least 90, at least 80, at least 70, at least 60, at least 50, at least 40, at least 35, at least 30, at least 25, at least 20, at least 15, at least 10, or at least 6 amino acid residues. Preferably, they comprise at least the amino acid sequence KX₁CGVX₂ (SEQ ID NO:104), wherein X₁ and X₂ can be any amino acid residue and X₁ and X₂ can be the same or different from one another. If the peptide comprises more than six amino acid residues, the amino acid residues adjacent to the amino acid sequence KX₁CGVX₂ (SEQ ID NO:104) may be any amino acid residues. Preferably, the adjacent amino acids are amino acid residues similar or identical to the amino acid residues being naturally adjacent to the sequence KLCGVL (SEQ ID NO:103) in a glycoprotein of a rabies virus strain. CR57 should still be capable of recognizing the peptides of the invention.

In an embodiment, the peptides of the invention can have a looped/cyclic form. Such peptides can be made by chemically converting the structures of linear peptides to looped/cyclic forms. It is well known in the art that cyclization of linear peptides can modulate bioactivity by increasing or decreasing the potency of binding to the target protein. Linear peptides are very flexible and tend to adopt many different conformations in solution. Cyclization acts to constrain the number of available conformations and, thus, favor the more active or inactive structures of the peptide. Cyclization of linear peptides is accomplished either by forming a peptide bond between the free N-terminal and C-terminal ends (homodetic cyclopeptides) or by forming a new covalent bond between amino acid backbone and/or side chain groups located near the N— or C-terminal ends (heterodetic cyclopeptides). The latter cyclizations use alternate chemical strategies to form covalent bonds, for example, disulfides, lactones, ethers, or thioethers. However, cyclization methods other than the ones described above can also be used to form cyclic/looped peptides. Generally, linear peptides of more than five residues can be cyclized relatively easily. The propensity of the peptide to form a beta-turn conformation in the central four residues facilitates the formation of both homo- and heterodetic cyclopeptides. The looped/cyclic peptides of the invention preferably comprise a cysteine residue at position 2 and 14. Preferably, they contain a linker between the cysteine residues. The looped/cyclic peptides of the invention are recognized by the human monoclonal antibodies described herein.

Alternatively, the peptides of the invention may be prepared by expression of the peptides or of a larger peptide including the desired peptide from a corresponding gene (whether synthetic or natural in origin) in a suitable host. The larger peptide may contain a cleavage site whereby the peptide of interest may be released by cleavage of the fused molecule.

The resulting peptides may then be tested for binding to at least one of the human monoclonal antibodies CR57, CRJA and CRJB, preferably CR57, in a way essentially as described herein. If such a peptide can still be bound by these antibodies, it is considered as a functional fragment or analogue of the peptides according to the invention. Also, even stronger antigenic peptides may be identified in this manner, which peptides may be used for vaccination purposes or for generating strongly neutralizing antibodies for therapeutic and/or prophylactic purposes. The peptides may even be used in diagnostic tests.

The invention also provides peptides comprising a part (or even consisting of a part) of a peptide according to the invention, wherein the part is recognized by at least one of the human monoclonal antibodies called CR57, CRJA and CRJB, preferably CR57. Preferably, the part recognized comprises the amino acid sequence KX₁CGVX₂ (SEQ ID NO:104).

Furthermore, the invention provides peptides consisting of an analogue of a peptide according to the invention, wherein one or more amino acids are substituted for another amino acid, and wherein the analogue is recognized by at least one of the human monoclonal antibodies called CR57, CRJA and CRJB, preferably CR57. Alternatively, analogues can be peptides of the present invention comprising an amino acid sequence containing insertions, deletions or combinations thereof of one or more amino acids compared to the amino acid sequences of the parent peptides. Furthermore, analogues can comprise truncations of the amino acid sequence at either or both the amino or carboxy termini of the peptides. Analogues according to the invention may have the same or different, either higher or lower, antigenic properties compared to the parent peptides, but are still recognized by at least one of the human monoclonal antibodies called CR57, CRJA and CRJB. That part of a 15-mer still representing immunogenic activity consists of about 6-12 residues within the 15-mer.

The peptides, parts thereof or analogues thereof according to the invention may be used directly as peptides, but may also be used conjugated to an immunogenic carrier, which may be, e.g., a polypeptide or polysaccharide. If the carrier is a polypeptide, the desired conjugate may be expressed as a fusion protein. Alternatively, the peptide and the carrier may be obtained separately and then conjugated. This conjugation may be covalently or non-covalently. A fusion protein is a chimeric protein, comprising the peptide according to the invention, and another protein or part thereof not being the rabies virus glycoprotein G. Such fusion proteins may, for instance, be used to raise antibodies for diagnostic, prophylactic and/or therapeutic purposes or to directly immunize, i.e., vaccinate, humans and/or animals. Any protein or part thereof or even peptide may be used as fusion partner for the peptides according to the invention to form a fusion protein, and non-limiting examples are bovine serum albumin, keyhole limpet hemocyanin, etc.

In another embodiment, the peptides of the invention may be comprised in a truncated G protein from a rhabdovirus, and even a lyssavirus, as herein described. Truncation/modification of proteins has been described above and is well within the reach of the skilled artisan.

The peptides may be labeled (signal-generating) or unlabeled. This depends on the type of assay used. Labels that may be coupled to the peptides are those known in the art and include, but are not limited to, enzymes, radionuclides, fluorogenic and chromogenic substrates, cofactors, biotin/avidin, colloidal gold, and magnetic particles.

It is another aspect of the invention to provide nucleic acid molecules encoding peptides, parts thereof or analogues thereof or encoding fusion proteins or conjugates according to the invention or encoding multimers of peptides according to the invention. Such nucleic acid molecules may suitably be used in the form of plasmids for propagation and expansion in bacterial or other hosts. Moreover, recombinant DNA techniques well known to the person skilled in the art can be used to obtain nucleic acid molecules encoding analogues of the peptides according to the invention, e.g., by mutagenesis of the sequences encoding the peptides according to the invention. One skilled in the art will appreciate that analogues of the nucleic acid molecules are also intended to be a part of the present invention. Analogues are nucleic acid sequences that can be directly translated, using the universal genetic code, to provide an amino acid sequence identical to that translated from the parent nucleic acid molecules. Another aspect of nucleic acid molecules according to the present invention is their potential for use in gene-therapy or vaccination applications. Therefore, in another embodiment of the invention, nucleic acid molecules according to the invention are provided wherein the nucleic acid molecule is present in a gene delivery vehicle. A “gene delivery vehicle” as used herein refers to an entity that can be used to introduce nucleic acid molecules into cells, and includes liposomes, naked DNA, plasmid DNA, optionally coupled to a targeting moiety such as an antibody with specificity for an antigen-presenting cell, recombinant viruses, bacterial vectors, and the like. Preferred gene therapy vehicles of the present invention will generally be viral vectors, such as comprised within a recombinant retrovirus, herpes simplex virus (HSV), adenovirus, adeno-associated virus (AAV), cytomegalovirus (CMV), and the like. Such applications of the nucleic acid sequences according to the invention are included in the present invention. The person skilled in the art will be aware of the possibilities of recombinant viruses for administering sequences of interest to cells. The administration of the nucleic acids of the invention to cells in vitro or in vivo can result in an enhanced immune response. Alternatively, the nucleic acid encoding the peptides of the invention can be used as naked DNA vaccines, e.g., immunization by injection of purified nucleic acid molecules into humans and/or animals or ex vivo.

In another aspect, the invention provides antibodies recognizing the peptides, parts or analogues thereof, fusion proteins or multimers of the invention. The peptides of the invention can be used for the discovery of a binding molecule, such as a human binding molecule such as a monoclonal antibody, whch upon binding to the peptide, reduces the infection of a host cell by a virus comprising the peptide. The antibodies according to the invention are not the three human monoclonal antibodies disclosed herein, i.e., CRJA, CRJB and CR57. Antibodies can be obtained according to routine methods well known to the person skilled in the art including, but not limited to, immunization of animals such as mice, rabbits, goats, and the like, or by antibody, phage or ribosome display methods (see e.g., Using Antibodies: A Laboratory Manual, edited by E. Harlow and D. Lane (1998), Cold Spring Harbor Laboratory Press, Cold Spring Harbor, N.Y.; Current Protocols in Immunology, edited by J. E. Coligan, A. M. Kruisbeek, D. H. Margulies, E. M. Shevach, W. Strober (2001), John Wiley & Sons Inc., New York; and Phage Display: A Laboratory Manual, edited by C. F. Barbas, D. R. Burton, J. K. Scott and G. J. Silverman (2001), Cold Spring Harbor Laboratory Press, Cold Spring Harbor, N.Y., the disclosures of which are incorporated herein by reference).

The antibodies of the invention can be intact immunoglobulin molecules such as polyclonal or monoclonal antibodies, in particular, human monoclonal antibodies, or the antibodies can be functional fragments thereof, i.e., fragments that are still capable of binding to the antigen. These fragments include, but are not limited to, Fab, F(ab′), F(ab′)2, Fv, dAb, Fd, complementarity-determining region (CDR) fragments, single-chain antibodies (scFv), bivalent single-chain antibodies, diabodies, triabodies, tetrabodies, and (poly)peptides that contain at least a fragment of an immunoglobulin that is sufficient to confer specific antigen binding to the (poly)peptides. The antibodies of the invention can be used in non-isolated or isolated form. Furthermore, the antibodies of the invention can be used alone or in a mixture/composition comprising at least one antibody (or variant or fragment thereof) of the invention. Antibodies of the invention include all the immunoglobulin classes and subclasses known in the art. Depending on the amino acid sequence of the constant domain of their heavy chains, binding molecules can be divided into the five major classes of intact antibodies: IgA, IgD, IgE, IgG, and IgM, and several of these may be further divided into subclasses (isotypes), e.g., IgA1, IgA2, IgG1, IgG2, IgG3 and IgG4. The above-mentioned antigen-binding fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, edited by E. Harlow and D. Lane (1988), Cold Spring Harbor Laboratory, Cold Spring Harbor, N.Y., which is incorporated herein by reference. A binding molecule or antigen-binding fragment thereof may have one or more binding sites. If there is more than one binding site, the binding sites may be identical to one another or they may be different.

The antibodies of the invention can be naked or unconjugated antibodies. A naked or unconjugated antibody is intended to refer to an antibody that is not conjugated, operatively linked or otherwise physically or functionally associated with an effector moiety or tag, such as, inter alia, a toxic substance, a radioactive substance, a liposome, an enzyme. It will be understood that naked or unconjugated antibodies do not exclude antibodies that have been stabilized, multimerized, humanized or in any other way manipulated, other than by the attachment of an effector moiety or tag. Accordingly, all post-translationally modified naked and unconjugated antibodies are included herewith, including where the modifications are made in the natural antibody-producing cell environment, by a recombinant antibody-producing cell, and are introduced by the hand of man after initial antibody preparation. Of course, the term naked or unconjugated antibody does not exclude the ability of the antibody to form functional associations with effector cells and/or molecules after administration to the body, as some of such interactions are necessary in order to exert a biological effect. The lack of associated effector group or tag is, therefore, applied in definition to the naked or unconjugated binding molecule in vitro, not in vivo.

Alternatively, the antibodies as described in the present invention can be conjugated to tags and be used for detection and/or analytical and/or diagnostic purposes. The tags used to label the antibodies for those purposes depend on the specific detection/analysis/diagnosis techniques and/or methods used, such as, inter alia, immunohistochemical staining of tissue samples, flow cytometric detection, scanning laser cytometric detection, fluorescent immunoassays, enzyme-linked immunosorbent assays (ELISAs), radioimmunoassays (RIAs), bioassays (e.g., neutralization assays, growth inhibition assays), Western blotting applications, etc. For immunohistochemical staining of tissue samples, preferred labels are enzymes that catalyze production and local deposition of a detectable product. Enzymes typically conjugated to antibodies to permit their immunohistochemical visualization are well known and include, but are not limited to, alkaline phosphatase, P-galactosidase, glucose oxidase, horseradish peroxidase, and urease. Typical substrates for production and deposition of visually detectable products include, but are not limited to, o-nitrophenyl-beta-D-galactopyranoside (ONPG), o-phenylenediamine dihydrochloride (OPD), p-nitrophenyl phosphate (PNPP), p-nitrophenyl-beta-D-galactopryanoside (PNPG), 3′, 3′diaminobenzidine (DAB), 3-amino-9-ethylcarbazole (AEC), 4-chloro-1-naphthol (CN), 5-bromo-4-chloro-3-indolyl-phosphate (BCIP), ABTS, BluoGal, iodonitrotetrazolium (INT), nitroblue tetrazolium chloride (NBT), phenazine methosulfate (PMS), phenolphthalein monophosphate (PMP), tetramethyl benzidine (TMB), tetranitroblue tetrazolium (TNBT), X-Gal, X-Gluc, and X-glucoside. Other substrates that can be used to produce products for local deposition are luminescent substrates. For example, in the presence of hydrogen peroxide, horseradish peroxidase can catalyze the oxidation of cyclic diacylhydrazides such as luminol. Next to that, binding molecules of the immunoconjugate of the invention can also be labeled using colloidal gold or they can be labeled with radioisotopes, such as ³³p, ³²p, ³⁵S, ³H, and ¹²⁵I. When the antibodies of the present invention are used for flow cytometric detections, scanning laser cytometric detections, or fluorescent immunoassays, they can usefully be labeled with fluorophores. A wide variety of fluorophores useful for fluorescently labeling the antibodies of the present invention include, but are not limited to, Alexa Fluor and Alexa Fluor&commat dyes, BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Cy2, Cy3, Cy3.5, CyS, Cy5.5, Cy7, fluorescein isothiocyanate (FITC), allophycocyanin (APC), R-phycoerythrin (PE), peridinin chlorophyll protein (PerCP), Texas Red, fluorescence resonance energy tandem fluorophores such as PerCP-Cy5.5, PE-Cy5, PE-Cy5.5, PE-Cy7, PE-Texas Red, and APC-Cy7. When the antibodies of the present invention are used for secondary detection using labeled avidin, streptavidin, captavidin or neutravidin, the antibodies may be labeled with biotin.

Next to that, the antibodies of the invention may be conjugated to photoactive agents or dyes such as fluorescent and other chromogens or dyes to use the so obtained immunoconjugates in photoradiation, phototherapy, or photodynamic therapy. The photoactive agents or dyes include, but are not limited to, photofrin.RTM, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series, chlorins, chlorin e6, mono-1-aspartyl derivative of chlorin e6, di-1-aspartyl derivative of chlorin e6, tin(IV) chlorin e6, meta-tetrahydroxyphenylchlor- in, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid benzonaphthoporphyrazines, cationic imminium salts, tetracyclines, lutetium texaphyrin, tin-etio-purpurin, porphycenes, benzophenothiazinium and combinations thereof.

When the antibodies of the invention are used for in vivo diagnostic use, the antibodies can also be made detectable by conjugation to, e.g., magnetic resonance imaging (MRI) contrast agents, such as gadolinium diethylenetriaminepentaacetic acid, to ultrasound contrast agents or to X-ray contrast agents, or by radioisotopic labeling.

Preferably, the antibodies according to the invention are capable of neutralizing rabies virus infectivity and are useful for therapeutic purposes against this virus. Assays to detect and measure virus neutralizing activity of antibodies are well known in the art and include, but are not limited to, the rapid fluorescent focus inhibition test (RFFIT), the mouse neutralization test (MNT), plaque assays, fluorescent antibody tests and enzyme immunoassays (Laboratory Techniques in Rabies, Chapter 15, pp. 181-192, edited by F.-X. Meslin, M. M. Kaplan, H. Koprowski (1996), World Health Organization).

Alternatively, the antibodies may inhibit or down-regulate rabies virus replication, are complement-fixing antibodies capable of assisting in the lysis of enveloped rabies virus and/or act as opsonins and augment phagocytosis of rabies virus, either by promoting its uptake via Fc or C3b receptors or by agglutinating rabies virus to make it more easily phagocytosed.

The invention also provides nucleic acid molecules encoding the antibodies according to the invention.

It is another aspect of the invention to provide vectors, i.e., nucleic acid constructs, comprising one or more nucleic acid molecules according to the present invention. The nucleic acid molecule may either encode the peptides, parts or analogues thereof or multimers or fusion proteins of the invention or encode the antibodies of the invention. Vectors can be derived from plasmids, such as, inter alia, F, R1, RP1, Col, pBR322, TOL, Ti, etc.; cosmids; phages such as lambda, lambdoid, M13, Mu, P1, P22, Qβ, T-even, T-odd, T2, T4, T7, etc.; plant viruses, such as, inter alia, alfalfa mosaic virus, bromovirus, capillovirus, carlavirus, carmovirus, caulivirus, clostervirus, comovirus, cryptovirus, cucumovirus, dianthovirus, fabavirus, fijivirus, furovirus, geminivirus, hordeivirus, ilarvirus, luteovirus, machlovirus, marafivirus, necrovirus, nepovirus, phytorepvirus, plant rhabdovirus, potexvirus, potyvirus, sobemovirus, tenuivirus, tobamovirus, tobravirus, tomato spotted wilt virus, tombusvirus, tymovirus, etc.; or animal viruses, such as, inter alia, adenovirus, arenaviridae, baculoviridae, birnaviridae, bunyaviridae, calciviridae, cardioviruses, coronaviridae, corticoviridae, cystoviridae, Epstein-Barr virus, enteroviruses, filoviridae, flaviviridae, Foot-and-Mouth disease virus, hepadnaviridae, hepatitis viruses, herpesviridae, immunodeficiency viruses, influenza virus, inoviridae, iridoviridae, orthomyxoviridae, papovaviruses, paramyxoviridae, parvoviridae, picomaviridae, poliovirus, polydnaviridae, poxviridae, reoviridae, retroviruses, rhabdoviridae, rhinoviruses, Semliki Forest virus, tetraviridae, togaviridae, toroviridae, vaccinia virus, vesicular stomatitis virus, etc. Vectors can be used for cloning and/or for expression of the peptides, parts or analogues thereof, of the invention, or antibodies of the invention and might even be used for gene therapy purposes. Vectors comprising one or more nucleic acid molecules according to the invention operably linked to one or more expression-regulating nucleic acid molecules are also covered by the present invention. The choice of vector is dependent on the recombinant procedures followed and the host used. Introduction of vectors in host cells can be effected by, inter alia, calcium phosphate transfection, virus infection, DEAE-dextran-mediated transfection, lipofectamin transfection or electroporation. Vectors may be autonomously replicating or may replicate together with the chromosome into which they have been integrated. Preferably, the vectors contain one or more selection markers. Useful markers are dependent on the host cells of choice and are well known to persons skilled in the art. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the peptides, parts or analogues thereof or antibodies as described above operably linked to one or more nucleic acid molecules encoding proteins or peptides that can be used to isolate these molecules are also covered by the invention. These proteins or peptides include, but are not limited to, glutathione-S-transferase, maltose-binding protein, metal-binding polyhistidine, green fluorescent protein, luciferase and beta-galactosidase.

Hosts containing one or more copies of the vectors mentioned above are an additional subject of the present invention. Preferably, the hosts are cells. Preferably, the cells are suitably used for the manipulation and propagation of nucleic acid molecules. Suitable cells include, but are not limited to, cells of mammalian, plant, insect, flngal or bacterial origin. Bacterial cells include, but are not limited to, cells from Gram-positive bacteria such as several species of the genera Bacillus, Streptomyces and Staphylococcus or cells of Gram-negative bacteria such as several species of the genera Escherichia, such as Escherichia coli, and Pseudomonas. In the group of flngal cells, preferably, yeast cells are used. Expression in yeast can be achieved by using yeast strains such as, inter alia, Pichia pastoris, Saccharomyces cerevisiae and Hansenula polymorpha. Furthermore, insect cells, such as cells from Drosophila and Sf9, can be used as host cells. Besides that, the host cells can be plant cells such as, inter alia, cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Preferably, the host cells are human cells. Examples of human cells are, inter alia, HeLa, 911, AT1080, A549, 293 and HEK293T cells. Preferred mammalian cells are human retina cells such as 911 cells or the cell line deposited at the European Collection of Cell Cultures (ECACC), CAMR, Salisbury, Wiltshire SP4 OJG, Great Britain on 29 Feb. 1996 under number 96022940 and marketed under the trademark PER.C6® (PER.C6 is a registered trademark of Crucell Holland B. V.). For the purposes of this application, “PER.C6” refers to cells deposited under number 96022940 or ancestors, passages up-stream or downstream, as well as descendants from ancestors of deposited cells, as well as derivatives of any of the foregoing.

PER.C6® cells can be used for the expression of antibodies to high levels (see, e.g., WO 00/63403) with human glycosylation patterns. The cells according to the invention may contain the nucleic acid molecule according to the invention in expressible format, such that the desired protein can be recombinantly expressed from the cells.

In a further aspect, the invention is directed to a peptide, part or analogue thereof according to the invention, or a fusion protein or conjugate according to the invention, or a multimer of peptides according to the invention, or a nucleic acid molecule encoding a peptide, part or analogue thereof according to the invention, or a nucleic acid molecule encoding a fusion protein or conjugate of the invention, or a nucleic acid molecule encoding a multimer of peptides according to the invention for use as a medicament. In other words, the invention is directed to a method of prevention and/or treatment wherein a peptide, part or analogue thereof according to the invention, or a fusion protein or conjugate according to the invention, or a multimer of peptides according to the invention, or a nucleic acid molecule encoding a peptide, part or analogue thereof according to the invention, or a nucleic acid molecule encoding a fusion protein or conjugate of the invention, or a nucleic acid molecule encoding a multimer of peptides according to the invention is used. Preferably, the peptides, parts or analogues thereof of the invention or molecules comprising these peptides, parts or analogues thereof may, for example, be for use as an immunogen, preferably a vaccine.

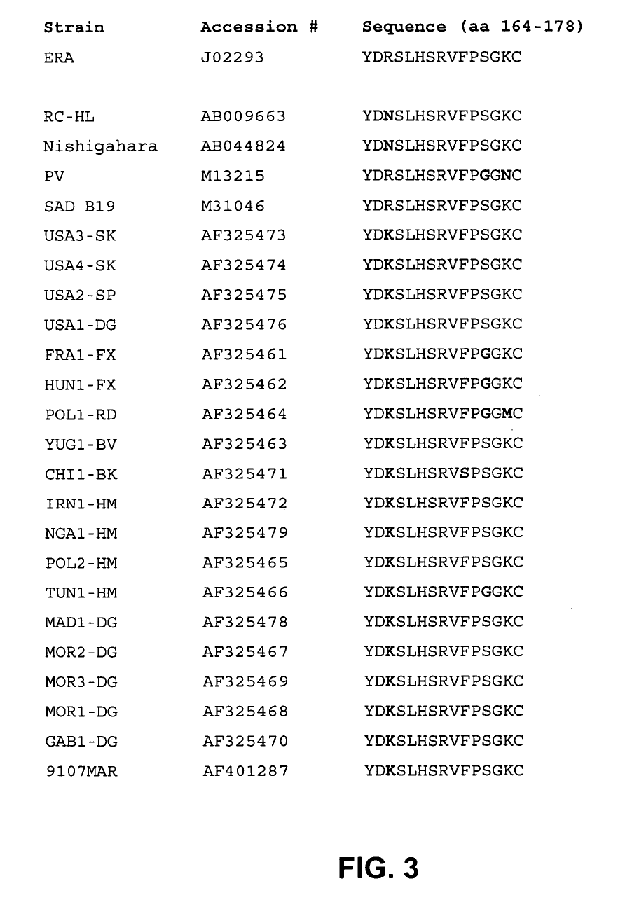

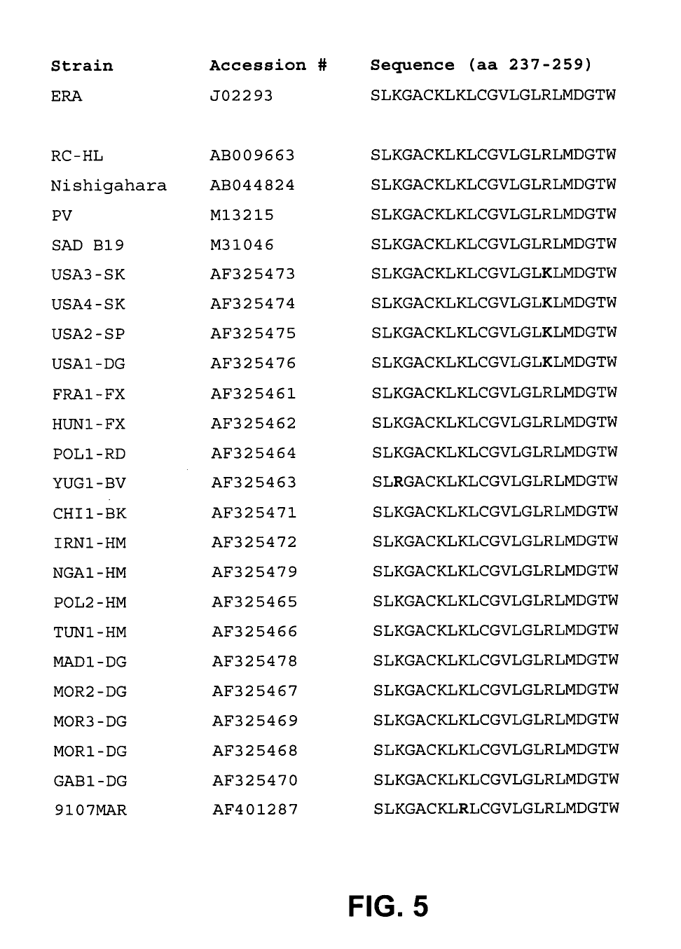

The antigenic peptides of the invention are obtained by binding of monoclonal anti-rabies virus antibodies to peptides prepared from the extracellular domain of glycoprotein G of the rabies virus strain ERA. The peptides may be useful in detection, prevention and/or treatment of a condition resulting from an infection with the rabies virus strain ERA. Numerous strains of rabies virus occur naturally. The glycoprotein G proteins of the various rabies strains are homologous to the glycoprotein G of strain ERA. The homology of the glycoprotein G proteins among genotype 1 varies between 90-99%. The extracellular domain of the glycoprotein G of rabies virus strain ERA is highly homologous to the extracellular domain of the glycoprotein G of other rabies virus strains. The homology of the extracellualr domain (without the signal sequence of amino acids 1- 19) of glycoprotein G proteins among genotype 1 varies between 92-99%. Interesting antigenic peptides are the peptides having the amino acid sequence selected from the group consisting of YDRSLHSRVFPSGKC (SEQ ID NO:2), SLKGACKLKLCGVLG (SEQ ID NO:6), LKGACKLKLCGVLGL (SEQ ID NO:7), KGACKLKLCGVLGLR (SEQ ID NO:8), GACKLKLCGVLGLRL (SEQ ID NO:9), ACKLKLCGVLGLRLM (SEQ ID NO:10), CKLKLCGVLGLRLMD (SEQ ID NO:11), KLKLCGVLGLRLMDG (SEQ ID NO:12), LKLCGVLGLRLMDGT (SEQ ID NO:13) and KLCGVLGLRLMDGTW (SEQ ID NO:14). The amino acid sequences of these peptides are identical or closely similar within the various rabies strains (see FIGS. 3 and 5). The core region or minimal binding region of the above peptides is the amino acid sequence KLCGVL (SEQ ID NO:103). This sequence (representing amino acids 226-231 of the mature rabies virus G protein of the ERA strain) is present in the G protein of a large number of rabies virus strains. In other words, the peptides of the invention do not differ in amino acid sequence, i.e., they are highly conserved, among strains of the rabies virus. Thus, a vaccine based on such peptides (derived from a single rabies virus strain, i.e., rabies virus strain ERA) may provide immunity in a vaccinated individual against other rabies virus strains. In other words, the vaccine will preferably be effective to provide protection against more strains of the rabies virus than vaccines of the prior art.

The peptides (or vaccines) may be administered to humans. However, as a means of rabies control, domesticated mammals, such as dogs, cats, horses, and cattle, may also be immunized against rabies virus by vaccination with these peptides. Furthermore, the peptides (or vaccines) may in theory even be used to immunize populations of wild animals, such as foxes, against rabies.

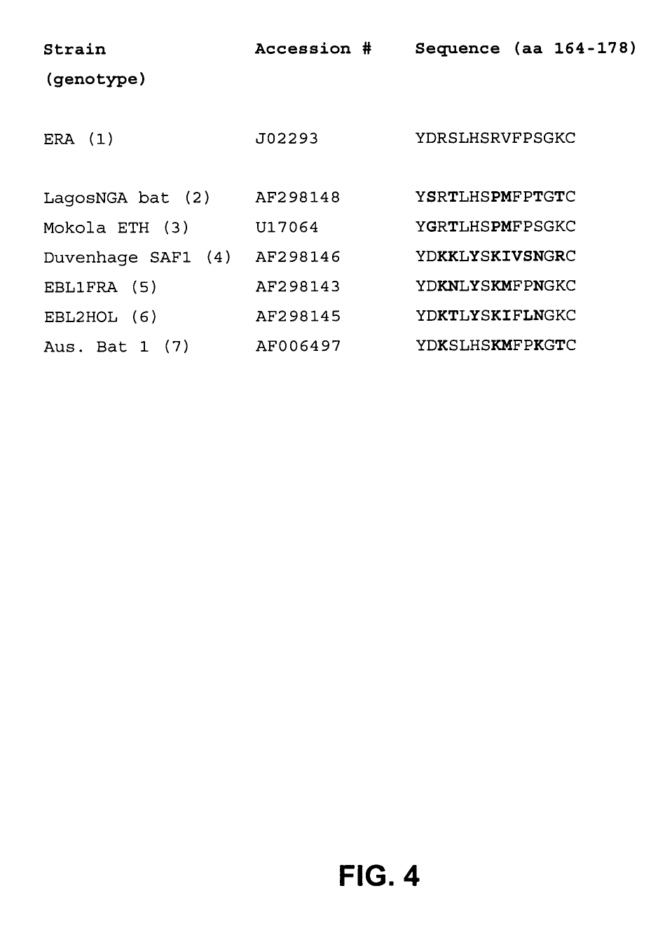

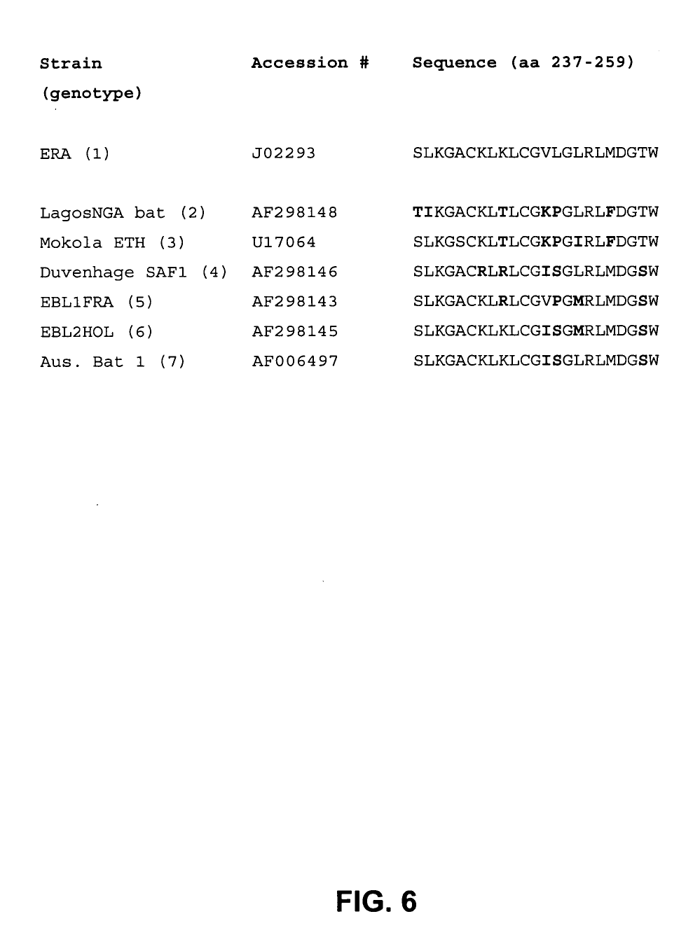

Rabies virus is part of the Lyssavirus genus. In total, the Lyssavirus genus includes seven genotypes: rabies virus (genotype 1), Lagos bat virus (genotype 2), Mokola virus (genotype 3), Duvenhage virus (genotype 4), European bat lyssavirus 1 (genotype 5), European bat lyssavirus 2 (genotype 6) and Australian bat lyssavirus (genotype 7). The peptides mentioned above are located in the region of amino acids 164-178 and 237-259 of the glycoprotein G of the rabies virus strain ERA. It might be possible that this similar position represents or harbors an antigenic region in surface glycoproteins of other Lyssavirus genera (see FIGS. 4 and 6 for amino acid sequences of these peptides). The peptide(s) in this region, in particular, peptides comprising the amino acid sequence KX₁CGVX₂ (SEQ ID NO:104), might therefore be useful in generating an immune response against other genotypes of the Lyssavirus genus. To investigate this, the peptide(s) present in this region could be synthesized and antibodies could be generated against the synthesized peptide(s). Techniques for synthesizing peptides and generating antibodies are well within the reach of the skilled artisan. Thereafter, it could be investigated if the obtained antibodies have neutralizing activity against the Lyssavirus strain from which the peptide(s) was/were obtained. The above strategy could also be followed for viruses of the rhabdovirus family. This family includes the genera cytorhabdovirus, ephemerovirus, lyssavirus, nucleorhabdovirus, rhabdovirus and vesiculovirus. As described above, it might be possible that peptides of viruses of the rhabdovirus family that are located at the similar position as the peptides of the glycoprotein G of the rabies virus strain ERA are antigenic peptides capable of inducing an immune response and giving protection against the rhabdovirus family viruses. The peptides (or vaccines) may also beneficially be used to immunize domesticated mammals and wild animals against viruses of the rhabdovirus family, particularly the Lyssavirus genus. Peptides have advantages compared to whole polypeptides when used as vaccines in that they are, for instance, easier to synthesize.

If the peptides, parts and analogues thereof of the invention are in the form of a vaccine, they are preferably formulated into compositions such as pharmaceutical compositions. A composition may also comprise more than one peptide of the invention. These peptides may be different or identical and may be linked, covalently or non-covalently, to each other or not linked to each other. For formulation of such (pharmaceutical) compositions, an immunogenically effective amount of at least one of the peptides of the invention is admixed with a physiologically acceptable carrier suitable for administration to animals including man. The peptides may be covalently attached to each other, to other peptides, to a protein carrier or to other carriers, incorporated into liposomes or other such vesicles, or complexed with an adjuvant or adsorbent as is known in the vaccine art. Alternatively, the peptides are not complexed with any of the above molecules and are merely admixed with a physiologically and/or pharmaceutically acceptable carrier such as normal saline or a buffering compound suitable for administration to animals including man. As with all immunogenic compositions for eliciting antibodies, the immunogenically effective amounts of the peptides of the invention must be determined. Factors to be considered include the immunogenicity of the native peptide, whether or not the peptide will be complexed with or covalently attached to an adjuvant or carrier protein or other carrier and route of administration for the composition, i.e., intravenous, intramuscular, subcutaneous, etc., and number of immunizing doses to be administered. Such factors are known in the vaccine art and it is well within the reach of a skilled artisan to make such determinations without undue experimentation. The peptides, parts or analogues thereof or compositions comprising these compounds may elicit an antibody response, preferably neutralizing antibody response, upon administering to human or animal subjects. Such an antibody response protects against further infection by rabies virus (or other viruses as described above) and/or will retard the onset or progress of the symptoms associated with rabies virus. In an embodiment, the peptides according to the invention can be used for the discovery of a binding molecule, such as a human binding molecule, that upon binding to the peptide, reduces the infection of a host cell by a virus such as a rhabdovirus comprising the peptide.

In yet another aspect, antibodies of the invention can be used as a medicament, preferably in the treatment of a condition resulting from rabies virus. In a specific embodiment, they can be used with any other medicament available to treat a condition resulting from rabies virus. In other words, the invention also pertains to a method of prevention and/or treatment, wherein the antibodies, fragments or functional variants thereof according to the invention are used. The antibodies might also be useful in the prevention and/or treatment of other rabies viruses, but also of viruses of the Lyssavirus genus or even of the rhabdovirus family. The antibodies of the invention can also be used for detection of rabies virus, but also of viruses of the Lyssavirus genus or even of the rhabdovirus family, e.g., for diagnostic purposes. Therefore, the invention provides a diagnostic test method for determining the presence of rabies virus in a sample, characterized in that the sample is put into contact with an antibody according to the invention. Preferably, the antibody is contacted with the sample under conditions which allow the formation of an immunological complex between the antibodies and rabies virus or fragments or (poly)peptides thereof that may be present in the sample. The formation of an immunological complex, if any, indicating the presence of rabies virus in the sample, is then detected and measured by suitable means. The sample may be a biological sample including, but not limited to, blood, serum, urine, tissue or other biological material from (potentially) infected subjects. The (potentially) infected subjects may be human subjects, but also animals that are suspected as carriers of rabies virus might be tested for the presence of rabies virus using these antibodies. Detection of binding may be according to standard techniques known to a person skilled in the art, such as an ELISA, Western blot, RIA, etc. The antibodies may suitably be included in kits for diagnostic purposes. It is, therefore, another aspect of the invention to provide a kit of parts for the detection of rabies virus comprising an antibody according to the invention. The antibodies of the invention may be used to purify rabies virus or a rabies virus fragment. Antibodies against peptides of the glycoprotein G of rabies virus may also be used to purify the protein or the extracellular domain thereof. Purification techniques for viruses and proteins are well known to the skilled artisan.

Also, the peptides of the invention might be used directly for the detection of rabies virus-recognizing antibodies, for instance, for diagnostic purposes. However, the antibodies are only recognized if they bind the specific peptides of the invention.

### EXAMPLES

### Example 1

Production of Human Monoclonal Antibodies CRJB, CRJA, CR57

First, the variable regions of mabs CR57, CRJB and CRJA were designed and synthesized. The cDNA sequences of the variable regions from the three anti-rabies mabs were transferred to GENEART. By means of software, GENEART has analyzed the sequences and suggested codon optimization strategies and sites for insertion of the appropriate restriction sites. The optimized sequences for the variable regions of the three mabs have been synthesized by GENEART. The SEQ ID NOS of the synthetic genes are shown in Table 1.

The nucleotide sequence of the redesigned variable regions of heavy and light chains of CR57 are shown in SEQ ID NO:20 and SEQ ID NO:22, respectively. The amino acid sequence of the redesigned variable regions of heavy and light chains of CR57 are shown in SEQ ID NO:21 and SEQ ID NO:23, respectively.

The nucleotide sequence of the redesigned variable regions of heavy and light chains of CRJA are shown in SEQ ID NO:24 and SEQ ID NO:26, respectively. The amino acid sequence of the redesigned variable regions of heavy and light chains of CRJA are shown in SEQ ID NO:25 and SEQ ID NO:27, respectively.

The nucleotide sequence of the redesigned variable regions of heavy and light chains of CRJB are shown in SEQ ID NO:28 and SEQ ID NO:30, respectively. The amino acid sequence of the redesigned variable regions of heavy and light chains of CRJB are shown in SEQ ID NO:29 and SEQ ID NO:31, respectively.

Next, the variable regions were cloned into synthetic vectors. The synthetic variable heavy region of monoclonal antibody CR57 was cloned into the synthetic IgG1 vector as follows. The variable region from SEQ ID NO:20 was cut with EcoRI and NheI and cloned into the EcoRI/NheI vector fragment of pcDNA-Sy-HCg1, resulting in pgCR57C03. The synthetic variable light region of monoclonal antibody CR57 was cloned into the synthetic lambda vector as follows. The variable region from SEQ ID NO:22 was cut with XhoI and HindIII and cloned into the XhoI/HindIII vector fragment of pcDNA-Sy-lambda, resulting in pgCR57C04. The synthetic variable heavy region of monoclonal antibody SOJA was cloned into the synthetic IgG1 vector as follows. The variable region from SEQ ID NO:24 was cut with EcoRI and NheI and cloned into the EcoRI/NheI vector fragment of pcDNA-Sy-HCg1, resulting in pgCRJAC03. The synthetic variable light region of monoclonal antibody CRJA was cloned into the synthetic kappa vector as follows. The variable region from SEQ ID NO:26 was cut with XhoI and RsrII and cloned into the XhoI/RsrII vector fragment of pcDNA-Sy-kappa, resulting in pgCRJAC05. The synthetic variable heavy region of monoclonal antibody CRJB was cloned into the synthetic IgG1 and vector as follows. The variable region from SEQ ID NO:28 was cut with EcoRI and NheI and cloned into the EcoRI/NheI vector fragment of pcDNA-Sy-HCg1 resulting in pgCRJBC03. The synthetic variable light region of monoclonal antibody CRJB was cloned into the synthetic kappa vector as follows. The variable region from SEQ ID NO:30 was cut with XhoI and HindIII and cloned into the XhoI/HindIII vector fragment of pcDNA-Sy-lambda, resulting in pgCRJBC04. All constructed vectors were checked for integrity by restriction enzyme analysis and DNA sequence analysis.

Next, the resulting expression constructs pgCR57C03, pgCRJAC03 and pgCRJBC03 encoding the anti-rabies human IgG1 heavy chains were transiently expressed in combination with the light chain expression constructs pgCR57C04, pgCRJAC05 and pgCRJBC04 in PER.C6® cells and supernatants containing IgG1 antibodies were obtained. The nucleotide sequences of the heavy chains of the antibodies called CR57, CRJA and CRJB are shown in SEQ ID NOS:32, 36, and 40, respectively. The amino acid sequences of the heavy chains of the antibodies called CR57, CRJA and CRJB are shown in SEQ ID NOS:33, 37 and 41, respectively.

The nucleotide sequences of the light chains of the antibodies called CR57, CRJA and CRJB are shown in SEQ ID NOS:34, 38, and 42, respectively. The amino acid sequences of the light chains of the antibodies called CR57, CRJA and CRJB are shown in SEQ ID NOS:35, 39, and 43, respectively.

Subsequently, the antibodies were purified over size-exclusion columns and protein-A columns using standard purification methods used generally for immunoglobulins (see, for instance, WO 00/63403).

### Example 2

PEPSCAN-ELISA

15-mer linear and looped/cyclic peptides were synthesized from the extracellular domain of the glycoprotein G of the rabies virus strain ERA (see FIG. 2 and SEQ ID NO:19 for the complete amino acid sequence of the glycoprotein G of the rabies virus strain ERA, the extracellular domain consists of amino acids 20-458; the protein-id of the glycoprotein of rabies virus strain ERA in the EMBL-database is AF406693) and screened using credit-card format mini-PEPSCAN cards (455 peptide formats/card) as described previously (Slootstra et al., 1996; WO 93/09872). All peptides were acetylated at the amino terminus.

In all looped peptides, position-2 and position-14 were replaced by a cysteine (acetyl-XCXXXXXXX XXXXCX-minicard). If other cysteines besides the cysteines at position-2 and position-14 were present in a prepared peptide, the other cysteines were replaced by an alanine. The looped peptides were synthesized using standard Fmoc-chemistry and deprotected using trifluoric acid with scavengers. Subsequently, the deprotected peptides were reacted on the cards with an 0.5 mM solution of 1,3-bis(bromomethyl)benzene in ammonium bicarbonate (20 mM, pH 7.9/acetonitril (1:1 (v/v)). The cards were gently shaken in the solution for 30 to 60 minutes, while completely covered in the solution. Finally, the cards were washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes.

The human monoclonal antibodies called CR57, CRJA and CRJB were prepared as described above. Binding of these antibodies to each linear and looped peptide was tested in a PEPSCAN-based enzyme-linked immuno assay (ELISA). The 455-well creditcard-format polypropylene cards, containing the covalently linked peptides, were incubated with the antibodies (10 μg/ml, with the exception of the PEPSCAN analysis following the alanine replacement scanning experiment wherein 100 μg/ml antibody was used; diluted in blocking solution which contains 5% horse-serum (v/v) and 5% ovalbumin (w/v)) (4° C., overnight). After washing, the peptides were incubated with anti-human antibody peroxidase (dilution 1/1000) (one hour, 25° C.), and subsequently, after washing the peroxidase substrate 2,2′-azino-di-3-ethylbenzthiazoline sulfonate (ABTS) and 2 μl/ml 3% H₂O₂ were added. Controls (for linear and looped) were incubated with anti-human antibody peroxidase only. After one hour, the color development was measured. The color development of the ELISA was quantified with a CCD-camera and an image processing system. The setup consists of a CCD-camera and a 55 mm lens (Sony CCD Video Camera XC-77RR, Nikon micro-nikkor 55 mm f/2.8 lens), a camera adaptor (Sony Camera adaptor DC-77RR) and the Image Processing Software package Optimas, version 6.5 (Media Cybernetics, Silver Spring, Md. 20910, U.S.A.). Optimas runs on a Pentium II computer system.

The human monoclonal antibodies called CR57, CRJA and CRJB were tested for binding to the 15-mer linear and looped/cyclic peptides synthesized as described supra. A peptide was considered to relevantly bind to an antibody when OD values were equal to or higher than two times the average OD value of all peptides (per antibody). See Table 2 for results of the binding of the human monoclonal antibodies called CR57, CRJA and CRJB to the linear peptides of the extracellular domain of glycoprotein G of rabies virus strain ERA.

Antibody CRJB (second column of Table 2) clearly bound to the linear peptide having the amino acid sequence YDRSLHSRVFPSGKC (SEQ ID NO:2).

Antibody CR57 (third column of Table 2) bound to the linear peptides having an amino acid sequence selected from the group consisting of YDRSLHSRVFPSGKC (SEQ ID NO:2), SLKGACKLKLCGVLG (SEQ ID NO:6), LKGACKLKLCGVLGL (SEQ ID NO:7), KGACKLKLCGVLGLR (SEQ ID NO:8), GACKLKLCGVLGLRL (SEQ ID NO:9), ACKLKLCGVLGLRLM (SEQ ID NO:10), CKLKLCGVLGLRLMD (SEQ ID NO:11), KLKLCGVLGLRLMDG (SEQ ID NO:12), LKLCGVLGLRLMDGT (SEQ ID NO:13) and KLCGVLGLRLMDGTW (SEQ ID NO:14). The peptides having the amino acid sequences GACKLKLCGVLGLRL (SEQ ID NO:9), ACKLKLCGVLGLRLM (SEQ ID NO:10) have an OD value that is lower than twice the average value. Nevertheless, these peptides were claimed because they are in the near proximity of a region of antigenic peptides recognized by antibody CR57. Binding was most prominent to the peptide with the amino acid sequence KLCGVLGLRLMDGTW (SEQ ID NO:14). This peptide, therefore, represents a good candidate of a hitherto unknown neutralizing epitope of rabies virus.

Antibody CRJA (fourth column of Table 2) clearly bound to the linear peptide having the amino acid sequence YDRSLHSRVFPSGKC (SEQ ID NO:2). This peptide was recognized by all three antibodies and, therefore, also represents a good candidate of a neutralizing epitope of rabies virus.

In Table 3, the relevant binding data of the three human monoclonal antibodies CRJB, CRJA and CR57 to the looped/cyclic peptides of the extracellular domain of the glycoprotein G of the rabies virus strain ERA are shown.

Antibody CRJB (second column of Table 3) clearly bound to the looped/cyclic peptide having an amino acid sequence selected from the group consisting of NHDYTIWMPENPRLG (SEQ ID NO:15) and WMPENPRLGMSCDIF (SEQ ID NO:5).

Antibody CR57 (third column of Table 3) clearly bound to the looped/cyclic peptide having an amino acid sequence selected from the group consisting of GYVTTTFKRKHFRPT (SEQ ID NO:1), YTIWMPENPRLGMSC (SEQ ID NO:3), IWMPENPRLGMSCDI (SEQ ID NO:4) and WMPENPRLGMSCDIF (SEQ ID NO:5).

Antibody CRJA (fourth column of Table 3) clearly bound to the looped/cyclic peptide having an amino acid sequence selected from the group consisting of DPYDRSLHSRVFPSG (SEQ ID NO:16), YCSTNHDYTIWMPEN (SEQ ID NO:17) and SFRRLSHLRKLVPGF (SEQ ID NO:18).

Any of the above peptides could form the basis for a vaccine or for raising neutralizing antibodies to treat and/or prevent a rabies virus infection. SLKGACKLKLCGVLGLRLMDGTW (SEQ ID NO:56) is a particularly interesting region of the glycoprotein based on its high reactivity in PEPSCAN. Linear peptides within this region clearly bound to the human monoclonal antibody called CR57. The specific region identified by PEPSCAN analysis might harbor a neutralizing epitope of the rabies glycoprotein. To confirm this, CVS-11 escape variants of CR57 were prepared and it was investigated as to whether these variants contained mutations in the region identified.

### Example 3

Interference of Selected Peptides with Antigen Binding of the CR57, CRJA and CRJB Antibodies

To further demonstrate that the selected peptides represent the neutralizing epitopes recognized by the antibodies called CR57, CRJA and CRJB, they are tested for their ability to interfere with binding of the CR57, CRJA and CRJB antibodies to the rabies glycoprotein. Interference of binding of the peptides of the invention is compared to interference of binding of irrelevant peptides. To this purpose, peptides of the invention are synthesized and solubilized. Subsequently, these peptides are incubated at increasing concentrations with 105 rabies glycoprotein-expressing 293T cells at 4° C. To this purpose, 293T cells are transiently transfected with an expression vector encoding the glycoprotein of the rabies virus ERA strain. Hereafter, the cells are stained with the antibodies called CR57, CRJA and CRJB. Staining of the antibodies is visualized using a phycoerithrin-labeled goat-anti-human IgG second step reagent(Caltag) and analyzed using flow cytometry according to methods known to a person skilled in the art.

### Example 4

Generation of Neutralization-Resistant Escape Viruses Using the CR57, CRJA and CRJB Antibody

To further analyze the epitopes that were recognized by the antibodies of above, neutralization-resistant escape variants of the rabies virus CVS-1 are selected in vitro. The escape variants are selected similarly as described by Lafon et al. 1983. In brief, serial ten-fold dilutions of virus are prepared using OPTI PRO SFM medium (GIBCO) containing ˜4 IU/ml monoclonal antibody. After an incubation of one hour at 37° C., 1 ml of the virus-antibody mixtures are added to monolayers of BSR cells grown in multidish 12 wells (Nunc) and the cells are incubated for three days at 34° C. After collecting the supernatants from the individual wells, the cells are fixed with 80% acetone, stained with FITC-labeled anti-rabies virus antibodies, and scored for fluorescent foci. Supernatants from the highest virus dilution still forming fluorescent foci are used to infect monolayers of BSR cells in T-25 flasks. The infected cells are replenished with OPTI PRO SFM medium (GIBCO) and incubated for three days at 34° C. The virus recovered from the T-25 flasks are used for virus neutralization tests. Using each antibody, five individual escape variants are isolated. A virus is defined as an escape variant if the neutralization index is less than 2.5 logs. The neutralization index is determined by subtracting the number of infectious virus particles/ml produced in BSR cell cultures infected with virus plus monoclonal antibody (˜4 IU/ml) from the number of infectious virus particles/ml produced in BSR cell cultures infected with virus alone (log focus forming units/ml virus in absence of monoclonal antibody minus log ffu/ml virus in presence of monoclonal antibody). An index lower than 2.5 logs is considered as evidence of escape. The isolated viruses are analyzed for mutations in their glycoprotein coding sequences. For this purpose, wild-type and escape variant viruses are purified by sucrose gradient ultracentrifugation and RNA is isolated from the purified virus. Glycoprotein cDNA is generated by RT-PCR using glycoprotein-specific oligonucleotides, the glycoprotein cDNA is sequenced using glycoprotein-specific sequencing primers.

Alternatively, neutralization-resistant escape viruses were prepared as follows. Serial ten-fold dilutions (0.5 ml; ranging from 10⁻¹ to 10⁻⁸) of virus were incubated with a constant amount (˜4 IU/ml) of monoclonal antibody CR57 or CRJB (0.5 ml) for one hour at 37° C./5% CO₂ before addition to monolayers of mouse neuroblastoma cells (MNA cells) or BSR cells (subclone of Baby Hamster Kidney cell line) grown in multidish 12 wells (Nunc). After three days of selection in the presence of CR57 or CRJB at 34° C./5% CO₂, medium (1 ml) containing potential escape viruses was harvested and stored at 4° C. until further use. Subsequently, the cells were fixed with 80% acetone, and stained overnight at 37° C./5% CO₂ with an anti-rabies N-FITC antibody conjugate (Centocor). The number of foci per well were scored by immunofluorescence and medium of wells containing one to six foci were chosen for virus amplification. Each escape virus was first amplified on a small scale on BSR or MNA cells depending on their growth characteristics. These small virus batches were then used to further amplify the virus on a large scale on MNA or BSR cells. Amplified virus was then titrated on MNA cells to determine the titer of each escape virus batch as well as the optimal dilution of the escape virus (giving 80-100% infection after 24 hours) for use in a virus neutralization assay.

For each of the antibodies CR57 and CRJB, six individual escape variants were isolated. A virus was defined as an escape variant if the neutralization index was <2.5 logs. The neutralization index was determined by subtracting the number of infectious virus particles/ml produced in BSR cell cultures infected with virus plus monoclonal antibody (˜4 IU/ml) from the number of infectious virus particles/ml produced in BSR or MNA cell cultures infected with virus alone (log focus forming units/ml virus in absence of monoclonal antibody minus log ffu/ml virus in presence of monoclonal antibody). An index lower than 2.5 logs was considered as evidence of escape.

Modified RFFIT (rapid fluorescent focus inhibition test) assays were performed to examine cross-protection of E57 (the escape viruses of CR57) and EJB (the escape viruses of CRJB) with CRJB and CR57, respectively. Therefore, CR57 or CRJB was diluted by serial three-fold dilutions starting with a 1:5 dilution. Rabies virus (strain CVS-11) was added to each dilution at a concentration that gives 80-100% infection. Virus/IgG mix was incubated for one hour at 37° C./5% CO₂ before addition to MNA cells. Twenty-four hours post-infection (at 34° C./5% CO₂), the cells were acetone-fixed for 20 minutes at 4° C., and stained for minimally three hours with an anti-rabies virus N-FITC antibody conjugate (Centocor). The wells were then analyzed for rabies virus infection under a fluorescence microscope to determine the 50% endpoint dilution. This is the dilution at which the virus infection is blocked by 50% in this assay. To calculate the potency, an international standard (Rabies Immune Globulin Lot R3, Reference material from the laboratory of Standards and Testing DMPQ/CBER/FDA) was included in each modified RFFIT. The 50% endpoint dilution of this standard corresponds with a potency of 2 IU/ml. The neutralizing potency of the single human monoclonal antibodies CR57 and CRJB, as well as the combination of these antibodies, were tested. EJB viruses were no longer neutralized by CRJB or CR57 (see Table 4), suggesting both antibodies bound to and induced amino acid changes in similar regions of the rabies virus glycoprotein. E57 viruses were no longer neutralized by CR57, whereas four out of six E57 viruses were still neutralized by CRJB, although with a lower potency (see Table 4). A mixture of the antibodies CR57 and CRJB (in a 1:1 IU/mg ratio) gave similar results as observed with the single antibodies (data not shown).

To identify possible mutations in the rabies virus glycoprotein, the nucleotide sequence of the glycoprotein open reading frame (ORF) of each of the EJB and E57 escape viruses was determined. Viral RNA of each of the escape viruses and CVS-11 was isolated from virus-infected MNA cells and converted into cDNA by standard RT-PCR. Subsequently, cDNA was used for nucleotide sequencing of the rabies virus glycoprotein ORFs in order to identify mutations.

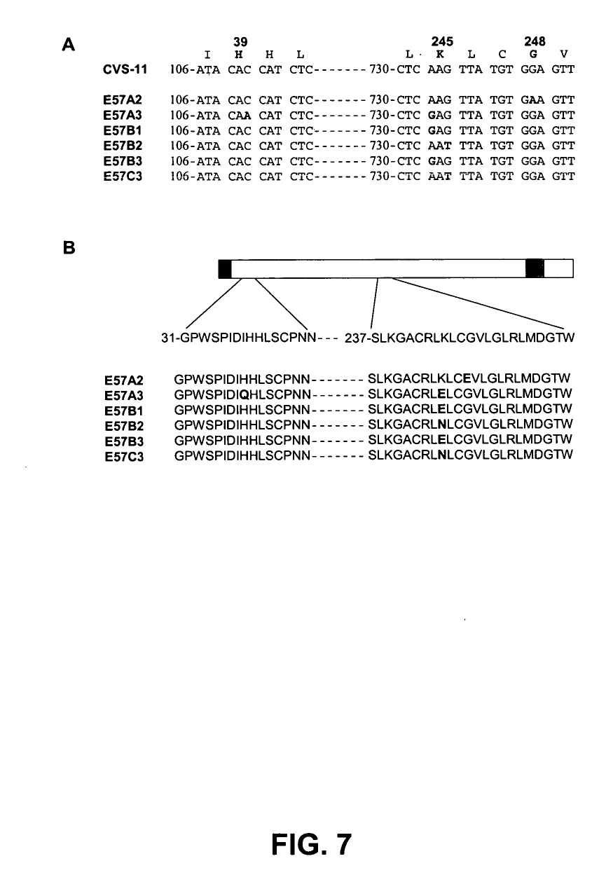

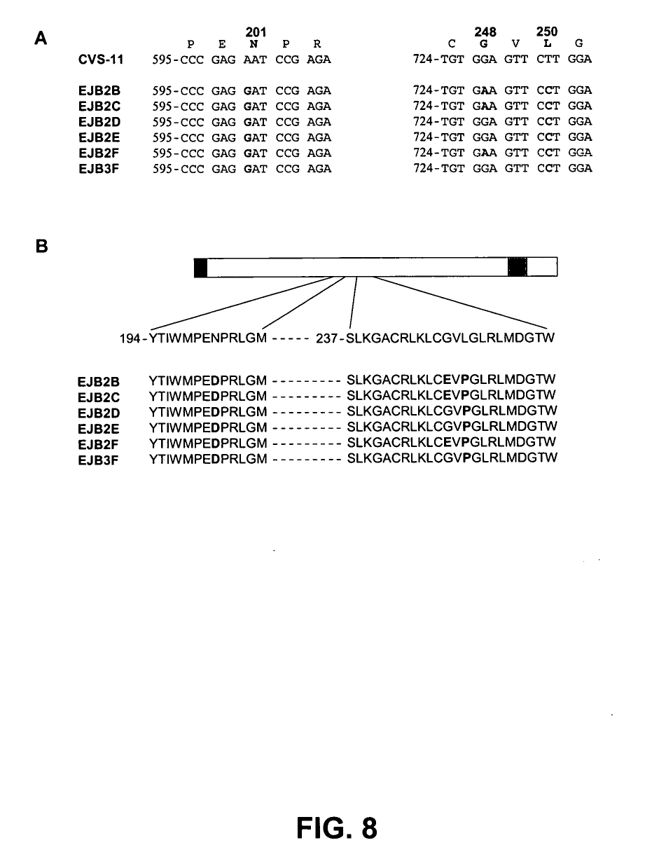

Both E57 and EJB escape viruses showed mutations in the region SLKGACKLKLCGVLGLRLMDGTW (SEQ ID NO:56) of the glycoprotein (see FIGS. 7 and 8). In addition to the PEPSCAN data showing that antibody CR57 binds to this specific region, this confirms that the region harbors a neutralizing epitope of the glycoprotein G. Moreover, a region having the amino acid sequence of YTIWMPENPRLGM (SEQ ID NO:83) appeared to be mutated in EJB escape viruses (substitution N→D; see FIG. 8). This might indicate that this region of the glycoprotein is together with the region SLKGACKLKLCGVLGLRLMDGTW (SEQ ID NO:56) part of a neutralizing epitope recognized by CRJB. Indeed, CRJB did display reactivity in the PEPSCAN analysis against looped/cyclic peptides (NHDYTIWMPENPRLG (SEQ ID NO:15); WMPENPRLGMSCDIF (SEQ ID NO:5)) spanning this region.

### Example 5

Determination of the CR57 Binding Region on Rabies Glycoprotein

PEPSCAN-ELISA essentially as described in Example 2 was performed to narrow down the neutralizing epitope recognized by CR57. 12-, 10-, and 8-mer peptides spanning SLKGACKLKLCGVLGLRLMDGTW (SEQ ID NO:56), i.e., the region shown to be reactive with CR57 (see Example 2) and shown to harbor a neutralizing epitope of rabies virus (see Example 4) were coupled as described before.

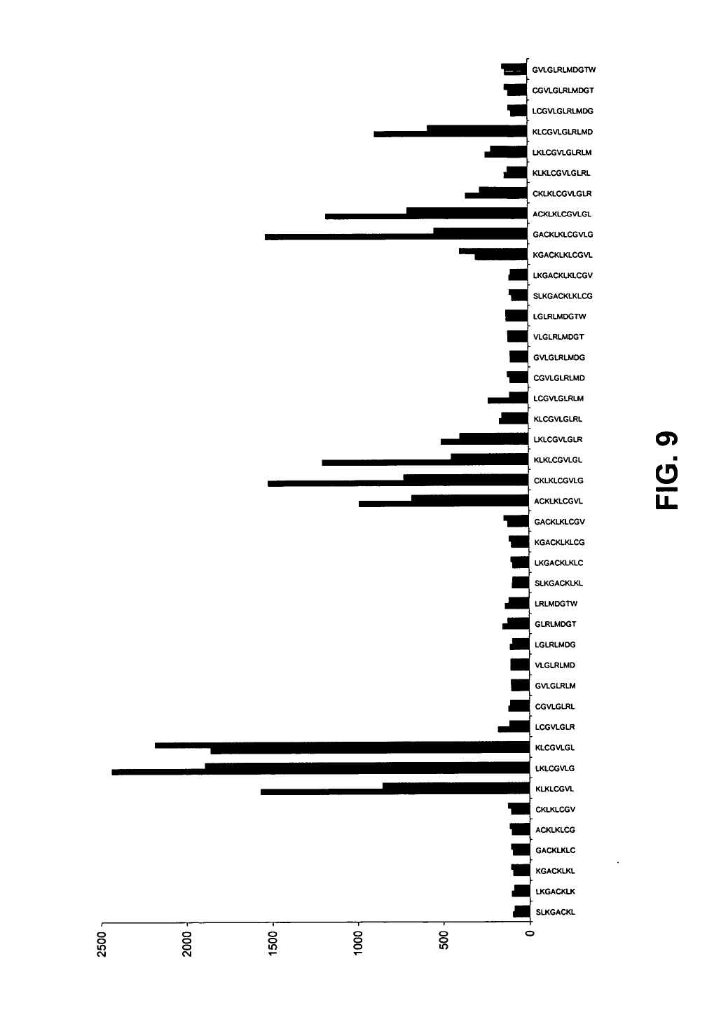

CR57 bound to the 12-mer peptides KGACKLKLCGVL (SEQ ID NO:88), GACKLKLCGVLG (SEQ ID NO:89), ACKLKLCGVLGL (SEQ ID NO:90), CKLKLCGVLGLR (SEQ ID NO:91), and KLCGVLGLRLMD (SEQ ID NO:92); to the 10-mer peptides ACKLKLCGVL (SEQ ID NO:93), CKLKLCGVLG (SEQ ID NO:94), KLKLCGVLGL (SEQ ID NO:95), and LKLCGVLGLR (SEQ ID NO:96); and to the 8-mer peptides KLKLCGVL (SEQ ID NO:97), LKLCGVLG (SEQ ID NO:98), and KLCGVLGL (SEQ ID NO:99) (see FIG. 9). Together, these data suggest that the epitope recognized by CR57 comprises the core region KLCGVL (SEQ ID NO:103). Furthermore, these results are in agreement with the amino acid mutations identified in the glycoprotein of each of the E57 escape viruses as shown in FIG. 7.

In addition, 12-, 10- and 8-mer peptides from the sequence SLKGACRLKLCGVLGLRLMDGTW (SEQ ID NO:74) were tested in PEPSCAN-ELISA. This amino acid sequence was identified from sequencing the glycoprotein ORF of the rabies virus strain wild-type CVS-11 (see FIG. 7). The sequence of the CVS-11 strain differs from the sequence of the ERA strain at one position (substitution K→R) in this region. Similar results as above were obtained with 12-, 10- and 8-mer peptides of the CVS-11 strain indicating that CR57 is capable of recognizing variant peptides (see FIG. 9). This also indicated that variations outside the core region of the neutralizing epitope do not interfere with the neutralization by CR57 of rabies virus strains harboring such sequence variations.

### Example 6

Epitope Mapping of CR57 on Rabies Glycoprotein

To determine the critical amino acids in the neutralizing epitope, an alanine scan (in combination with PEPSCAN-ELISA) was performed on three peptides (LKLCGVLG (SEQ ID NO:98), KLCGVLGLRLMD (SEQ ID NO:92), GACKLKLCGVLG (SEQ ID NO:89)) shown to be reactive with CR57 (see Example 5). In the alanine replacement scan, single alanine mutations were introduced at every residue contained with the above-mentioned peptides. In case an alanine was already present in the peptide, this alanine was mutated into a glycine.

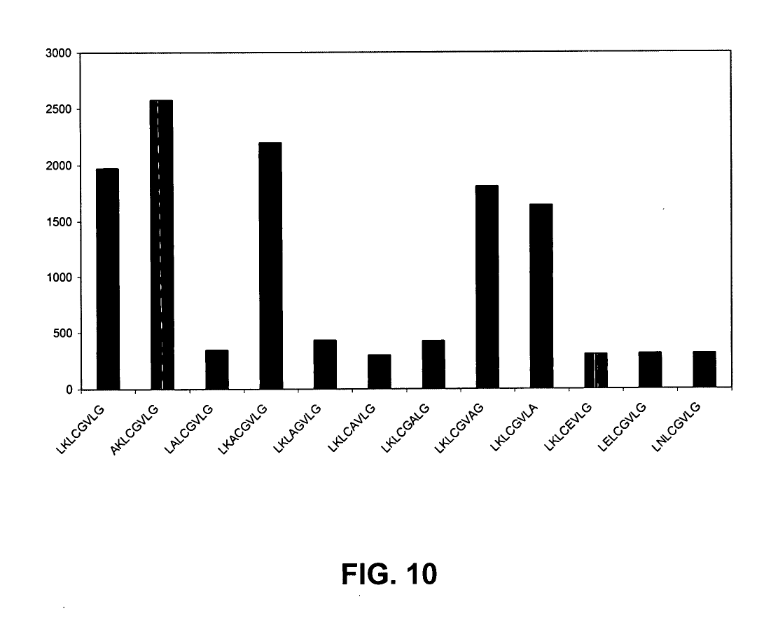

FIG. 10 shows the alanine replacement scan of peptide LKLCGVLG (SEQ ID NO:98). From FIG. 10, it can be determined that antibody CR57 is no longer reactive with the peptides having the amino acid sequence LALCGVLG (SEQ ID NO:109), LKLAGVLG (SEQ ID NO:110), LKLCAVLG (SEQ ID NO:111) and LKLCGALG (SEQ ID NO:112). Similar results were also obtained with the longer peptides on which an alanine replacement scan was performed (data not shown). Together, the above results revealed the critical residues of the neutralizing epitope, particularly the core region of the epitope, i.e., KLCGVL (SEQ ID NO:103), important for binding of CR57. The amino acids of the core region critical for binding of CR57 are K, C, G and V. In view thereof, the amino acid sequence of the core region sufficient for binding appears to be KX₁CGVX₂ (SEQ ID NO:104).

In addition, the 8-mer peptides LELCGVLG (SEQ ID NO:100, LNLCGVLG (SEQ ID NO:101) and LKLCEVLG (SEQ ID NO:102) harboring the mutations observed in the epitope in E57 escape viruses (see FIG. 7) were synthesized and tested by means of PEPSCAN-ELISA to confirm the effect of these mutations on binding and neutralization. In FIG. 10 it is shown that LELCGVLG (SEQ ID NO:100, LNLCGVLG (SEQ ID NO:101) and LKLCEVLG (SEQ ID NO:102) were no longer reactive with antibody CR57. Lack of binding of CR57 to the peptides comprising the mutations further confirmed the observed lack of neutralization by CR57 of E57 escape viruses (see Example 4).

As indicated above, the epitope recognized by CR57 comprises the minimal binding region having the amino acid sequence KLCGVL (SEQ ID NO:103). This sequence (representing amino acids 245-250 of the rabies virus G protein of the ERA strain) is present in the G protein of a large number of rabies virus strains. Alignment of the minimal binding regions of 229 genotype 1 rabies virus isolates was performed to assess the conservation of the epitope. The alignment sample set contained human isolates, bat isolates, and isolates from canines or from domestic animals most likely bitten by rabid canines. The minimal binding region of the epitope was aligned using glycoprotein sequences of the following 229 rabies virus isolates: AY353900, AY353899, AY353898, AY353897, AY353896, AY353895, AY353894, AY353893, AY353892, AY353867, AY353891, AY353889, AY353888, AY353887, AY353886, AY353885, AY353884, AY353883, AY353882, AY353881, AY353880, AY353879, AY353878, AY353877, AY353876, AY353875, AY353874, AY353873, AY353872, AY353871, AY353870, AY353869, AY353866, AY353868, AY353865, AY353864, AY353863, AY353862, AY353861, AY353860, AY353859, AY353858, AY353857, AB110669, AB110668, AB110667, AB110666, AB110665, AB110664, AB110663, AB110662, AB110661, AB110660, AB110659, AB110658, AB110657, AB110656, AY257983, AY257982, AY170424, AY170423, AY170422, AY170421, AY170420, AY170419, AY170418, AY257981, AY257980, AB115921, AY237121, AY170438, AY170437, AY170436, AY170435, AY170434, AY170433, AY170432, AY170431, AY170430, AY170429, AY170428, AY170427, AY170426, AY170425, U72051, U72050, U72049, AY103017, AY103016, AF298141, AF401287, AF401286, AF401285, AF134345, AF134344, AF134343, AF134342, AF134341, AF134340, AF134339, AF134338, AF134337, AF134336, AF134335, AF134334, AF134333, AF134332, AF134331, AF134330, AF134329, AF134328, AF134327, AF134326, AF134325, AF233275, AF325495, AF325494, AF325493, AF325492, AF325491, AF325490, AF325489, AF325488, AF325487, AF325486, AF325485, AF325484, AF325483, AF325482, AF325481, AF325480, AF325479, AF325478, AF325477, AF325476, AF325475, AF325474, AF325473, AF325472, AF325471, AF325470, AF325469, AF325468, AF325467, AF325466, AF325465, AF325464, AF325463, AF325462, AF325461, AF346891, AF326890, AF346889 AF346888, AF346887, AF346886, AF346885, AF346884, AF346883, AF346882, AF346881, AF346880, AF346879, AF346878, AF346877, AF346876, AF346875, AF346874, AF346873, AF346872, AF346871, AF346870, AF346869, AF346868, AF346867, AF346866, AF346865, AF346864, AF346863, AF346862, AF346861, AF346860, AF346859, AF346858, AF346857, AF346856, AF346855, AF344307, AF344305, U11756, U11752, U11751, U11750, U11748, U11747, U11746, U11745, U11744, U11743, U11742, U11741, U11739, U11737, U11736, U27217, U27216, U27215, U27214, U11758, U11757, U11755, U11754, U11753, AB052666, AY009100, AY009099, AY009098, AY009097, AH007057, U52947, U52946, U03767, U03766, U03765, U03764, L04523, M81058, M81059, M81060. Frequency analysis of the amino acids at each position within the minimal binding region revealed that the critical residues constituting the epitope were highly conserved. The lysine at position one was conserved in 99.6% of the isolates, while in only one of the 229 isolates, a conservative K >R mutation was observed. Positions two and three (L and C) were completely conserved. The glycine at position four was conserved in 98.7% of the isolates, while in three of the 229 isolates, mutations towards charged amino acids (G>R in one isolate and G>E in two isolates) were observed. The fifth position was also conserved with the exception of one isolate where a conservative V>I mutation was observed. At the sixth position, which is not a critical residue, significant heterogeneity is observed in the street isolates. A leucine is found in 70.7%, a proline in 26.7% and a serine in 2.6% of the isolates. The occurrence of amino acids at the various positions of the minimal binding region is depicted in Table 5. From the 229 analyzed naturally occurring rabies virus isolates, only three isolates (AF346857, AF346861, U72050) contained non-conserved amino acid changes at key residues within the epitope that would abrogate antibody binding. In two bat virus isolates (AF346857, AF346861), the amino acid changes within the epitope were identical to those observed in some of the EJB viruses (i.e., KLCEVP (SEQ ID NO:113)). However, none of the 229 rabies virus isolates contained an aspartic acid at position 182 of the mature glycoprotein as was observed in the EJB viruses.

TABLE 1
SEQ ID NOs of nucleotide and amino acid sequences
of synthetic variable regions and complete
heavy and light chains of anti-rabies mabs

| | Synthetic | complete | Synthetic | complete |
|---|---|---|---|---|
| mAb | VH | heavy chain | VL | light chain |
| CR57 DNA | SEQ ID 20 | SEQ ID 32 | SEQ ID 22 | SEQ ID 34 |
| prt | SEQ ID 21 | SEQ ID 33 | SEQ ID 23 | SEQ ID 35 |
| CRJA DNA | SEQ ID 24 | SEQ ID 36 | SEQ ID 26 | SEQ ID 38 |
| prt | SEQ ID 25 | SEQ ID 37 | SEQ ID 27 | SEQ ID 39 |
| CRJB DNA | SEQ ID 28 | SEQ ID 40 | SEQ ID 30 | SEQ ID 42 |
| prt | SEQ ID 29 | SEQ ID 41 | SEQ ID 31 | SEQ ID 43 |

TABLE 2

Binding of the human monoclonal antibodies CRJB,
CRJA CR57 to linear peptides of the extracellular
domain of glycoprotein G of rabies virus strain
ERA.

| | | | | |
|---|---|---|---|---|
| Amino acid | | | | |
| sequence | CRJB | CR57 | CRJA | SEQ |
| of linear | (10 μg/ | (10 μg/ | (10 μg/ | ID |
| peptide | ml) | ml) | ml) | NO |

| | | | | |
|---|---|---|---|---|
| KFPIYTILDKLGPWS | 97 | 71 | 65 | 114 |
| FPIYTILDKLGPWSP | 105 | 42 | 88 | 115 |
| PIYTILDKLGPWSPI | 89 | 36 | 143 | 116 |
| IYTILDKLGPWSPID | 97 | 44 | 83 | 117 |
| YTILDKLGPWSPIDI | 114 | 48 | 93 | 118 |
| TILDKLGPWSPIDIH | 96 | 76 | 84 | 119 |
| ILDKLGPWSPIDIHH | 104 | 54 | 56 | 120 |
| LDKLGPWSPIDIHHL | 99 | 55 | 59 | 121 |
| DKLGPWSPIDIHHLS | 103 | 62 | 78 | 122 |
| KLGPWSPIDIHHLSC | 105 | 72 | 72 | 123 |
| LGPWSPIDIHHLSCP | 112 | 69 | 84 | 124 |
| GPWSPIDIHHLSCPN | 114 | 68 | 72 | 125 |
| PWSPIDIHHLSCPNN | 104 | 62 | 76 | 126 |
| WSPIDIHHLSCPNNL | 106 | 80 | 83 | 127 |
| SPIDIHHLSCPNNLV | 85 | 74 | 100 | 128 |
| PIDIHHLSCPNNLVV | 93 | 46 | 39 | 129 |
| IDIHHLSCPNNLVVE | 102 | 69 | 61 | 130 |
| DIHHLSCPNNLVVED | 96 | 38 | 61 | 131 |
| IHHLSCPNNLVVEDE | 85 | 37 | 79 | 132 |
| HHLSCPNNLVVEDEG | 76 | 56 | 72 | 133 |
| HLSCPNNLVVEDEGC | 119 | 65 | 76 | 134 |
| LSCPNNLVVEDEGCT | 117 | 69 | 90 | 135 |
| SCPNNLVVEDEGCTN | 114 | 83 | 88 | 136 |
| CPNNLVVEDEGCTNL | 97 | 77 | 75 | 137 |
| PNNLVVEDEGCTNLS | 107 | 78 | 86 | 138 |
| NNLVVEDEGCTNLSG | 99 | 72 | 93 | 139 |
| NLVVEDEGCTNLSGF | 119 | 75 | 85 | 140 |
| LVVEDEGCTNLSGFS | 103 | 76 | 58 | 141 |
| VVEDEGCTNLSGFSY | 107 | 73 | 63 | 142 |
| VEDEGCTNLSGFSYM | 103 | 74 | 82 | 143 |
| EDEGCTNLSGFSYME | 90 | 54 | 65 | 144 |
| DEGCTNLSGFSYMEL | 23 | 1 | 54 | 145 |
| EGCTNLSGFSYMELK | 114 | 51 | 59 | 146 |
| GCTNLSGFSYMELKV | 114 | 55 | 72 | 147 |
| CTNLSGFSYMELKVG | 110 | 47 | 84 | 148 |
| TNLSGFSYMELKVGY | 106 | 43 | 102 | 149 |
| NLSGFSYMELKVGYI | 115 | 61 | 94 | 150 |
| LSGFSYMELKVGYIL | 132 | 71 | 82 | 151 |
| SGFSYMELKVGYILA | 132 | 79 | 105 | 152 |
| GFSYMELKVGYILAI | 111 | 65 | 91 | 153 |
| FSYMELKVGYILAIK | 112 | 89 | 120 | 154 |
| SYMELKVGYILAIKM | 123 | 65 | 143 | 155 |
| YMELKVGYILAIKMN | 114 | 78 | 96 | 156 |
| MELKVGYILAIKMNG | 141 | 76 | 92 | 157 |
| ELKVGYILAIKMNGF | 132 | 87 | 84 | 158 |
| LKVGYILAIKMNGFT | 112 | 78 | 68 | 159 |
| KVGYILAIKMNGFTC | 118 | 78 | 83 | 160 |
| VGYILAIKMNGFTCT | 93 | 77 | 70 | 161 |
| GYILAIKMNGFTCTG | 90 | 75 | 73 | 162 |
| YILAIKMNGFTCTGV | 107 | 47 | 45 | 163 |
| ILAIKMNGFTCTGVV | 103 | 79 | 87 | 164 |
| LAIKMNGFTCTGVVT | 130 | 68 | 112 | 165 |
| AIKMNGFTCTGVVTE | 103 | 47 | 93 | 166 |
| IKMNGFTCTGVVTEA | 108 | 68 | 88 | 167 |
| KMNGFTCTGVVTEAE | 104 | 76 | 90 | 168 |
| MNGFTCTGVVTEAEN | 99 | 69 | 87 | 169 |
| NGFTCTGVVTEAENY | 101 | 69 | 98 | 170 |
| GFTCTGVVTEAENYT | 86 | 71 | 90 | 171 |
| FTCTGVVTEAENYTN | 125 | 83 | 91 | 172 |
| TCTGVVTEAENYTNF | 112 | 92 | 96 | 173 |
| CTGVVTEAENYTNFV | 123 | 76 | 89 | 174 |
| TGVVTEAENYTNFVG | 110 | 85 | 86 | 175 |
| GVVTEAENYTNFVGY | 111 | 86 | 76 | 176 |
| VVTEAENYTNFVGYV | 106 | 87 | 90 | 177 |
| VTEAENYTNFVGYVT | 90 | 79 | 79 | 178 |
| TEAENYTNFVGYVTT | 84 | 68 | 86 | 179 |
| EAENYTNFVGYVTTT | 117 | 69 | 62 | 180 |
| AENYTNFVGYVTTTF | 106 | 66 | 74 | 181 |
| ENYTNFVGYVTTTFK | 112 | 44 | 80 | 182 |
| NYTNFVGYVTTTFKR | 114 | 49 | 97 | 183 |
| YTNFVGYVTTTFKRK | 104 | 51 | 76 | 184 |
| TNFVGYVTTTFKRKH | 125 | 71 | 96 | 185 |
| NFVGYVTTTFKRKHF | 107 | 65 | 88 | 186 |
| FVGYVTTTFKRKHFR | 111 | 70 | 79 | 187 |
| VGYVTTTFKRKHFRP | 113 | 75 | 80 | 188 |
| GYVTTTFKRKHFRPT | 123 | 70 | 87 | 1 |
| YVTTTFKRKHFRPTP | 106 | 85 | 84 | 189 |
| VTTTFKRKHFRPTPD | 105 | 79 | 77 | 190 |
| TTTFKRKHFRPTPDA | 108 | 80 | 76 | 191 |
| TTFKRKHFRPTPDAC | 99 | 74 | 111 | 192 |
| TFKRKHFRPTPDACR | 111 | 96 | 97 | 193 |
| FKRKHFRPTPDACRA | 92 | 64 | 86 | 194 |
| KRKHFRPTPDACRAA | 93 | 65 | 65 | 195 |
| RKHFRPTPDACRAAY | 107 | 64 | 57 | 196 |
| KHFRPTPDACRAAYN | 112 | 73 | 85 | 197 |
| HFRPTPDACRAAYNW | 113 | 46 | 93 | 198 |
| FRPTPDACRAAYNWK | 112 | 43 | 104 | 199 |
| RPTPDACRAAYNWKM | 101 | 77 | 123 | 200 |
| PTPDACRAAYNWKMA | 125 | 99 | 129 | 201 |
| TPDACRAAYNWKMAG | 132 | 92 | 132 | 202 |
| PDACRAAYNWKMAGD | 124 | 61 | 93 | 203 |
| DACRAAYNWKMAGDP | 113 | 84 | 83 | 204 |
| ACRAAYNWKMAGDPR | 116 | 82 | 93 | 205 |
| CRAAYNWKMAGDPRY | 118 | 87 | 113 | 206 |
| RAAYNWKMAGDPRYE | 130 | 90 | 92 | 207 |
| AAYNWKMAGDPRYEE | 106 | 68 | 78 | 208 |
| AYNWKMAGDPRYEES | 94 | 96 | 90 | 209 |
| YNWKMAGDPRYEESL | 118 | 83 | 110 | 210 |
| NWKMAGDPRYEESLH | 101 | 58 | 69 | 211 |
| WKMAGDPRYEESLHN | 101 | 69 | 86 | 212 |
| KMAGDPRYEESLHNP | 102 | 62 | 48 | 213 |
| MAGDPRYEESLHNPY | 116 | 64 | 71 | 214 |
| AGDPRYEESLHNPYP | 101 | 40 | 83 | 215 |
| GDPRYEESLHNPYPD | 98 | 36 | 96 | 216 |
| DPRYEESLHNPYPDY | 110 | 57 | 92 | 217 |
| PRYEESLHNPYPDYR | 115 | 73 | 103 | 218 |
| RYEESLHNPYPDYRW | 112 | 69 | 96 | 219 |
| YEESLHNPYPDYRWL | 106 | 58 | 87 | 220 |
| EESLHNPYPDYRWLR | 123 | 76 | 85 | 221 |
| ESLHNPYPDYRWLRT | 132 | 92 | 80 | 222 |
| SLHNPYPDYRWLRTV | 111 | 78 | 87 | 223 |
| LHNPYPDYRWLRTVK | 106 | 79 | 86 | 224 |
| HNPYPDYRWLRTVKT | 108 | 86 | 98 | 225 |
| NPYPDYRWLRTVKTT | 102 | 85 | 106 | 226 |
| PYPDYRWLRTVKTTK | 93 | 65 | 84 | 227 |
| YPDYRWLRTVKTTKE | 97 | 72 | 88 | 228 |
| PDYRWLRTVKTTKES | 85 | 76 | 83 | 229 |
| DYRWLRTVKTTKESL | 111 | 54 | 55 | 230 |
| YRWLRTVKTTKESLV | 117 | 46 | 68 | 231 |
| RWLRTVKTTKESLVI | 110 | 40 | 72 | 232 |
| WLRTVKTTKESLVII | 104 | 41 | 85 | 233 |
| LRTVKTTKESLVIIS | 104 | 65 | 83 | 234 |
| RTVKTTKESLVIISP | 120 | 82 | 103 | 235 |
| TVKTTKESLVIISPS | 116 | 76 | 93 | 236 |
| VKTTKESLVIISPSV | 120 | 71 | 96 | 237 |
| KTTKESLVIISPSVA | 112 | 101 | 82 | 238 |
| TTKESLVIISPSVAD | 121 | 78 | 91 | 239 |
| TKESLVIISPSVADL | 112 | 86 | 102 | 240 |
| KESLVIISPSVADLD | 117 | 86 | 123 | 241 |
| ESLVIISPSVADLDP | 125 | 88 | 120 | 242 |
| SLVIISPSVADLDPY | 105 | 68 | 88 | 243 |
| LVIISPSVADLDPYD | 107 | 85 | 104 | 244 |
| VIISPSVADLDPYDR | 98 | 59 | 47 | 245 |
| IISPSVADLDPYDRS | 125 | 83 | 98 | 246 |
| ISPSVADLDPYDRSL | 119 | 50 | 56 | 247 |
| SPSVADLDPYDRSLH | 114 | 59 | 72 | 248 |
| PSVADLDPYDRSLHS | 114 | 44 | 72 | 249 |
| SVADLDPYDRSLHSR | 106 | 49 | 92 | 250 |
| VADLDPYDRSLHSRV | 113 | 71 | 92 | 251 |
| ADLDPYDRSLHSRVF | 121 | 70 | 100 | 252 |
| DLDPYDRSLHSRVFP | 152 | 111 | 107 | 253 |
| LDPYDRSLHSRVFPS | 142 | 99 | 113 | 254 |
| DPYDRSLHSRVFPSG | 120 | 90 | 92 | 16 |
| PYDRSLHSRVFPSGK | 120 | 86 | 104 | 255 |
| YDRSLHSRVFPSGKC | 818 | 364 | 1027 | 2 |
| DRSLHSRVFPSGKCS | 142 | 98 | 187 | 256 |
| RSLHSRVFPSGKCSG | 141 | 87 | 125 | 257 |
| SLHSRVFPSGKCSGV | 111 | 69 | 96 | 258 |
| LHSRVFPSGKCSGVA | 114 | 78 | 134 | 259 |
| HSRVFPSGKCSGVAV | 118 | 97 | 111 | 260 |
| SRVFPSGKCSGVAVS | 125 | 100 | 107 | 261 |
| RVFPSGKCSGVAVSS | 110 | 69 | 58 | 262 |
| VFPSGKCSGVAVSST | 114 | 74 | 68 | 263 |
| FPSGKCSGVAVSSTY | 134 | 64 | 93 | 264 |
| PSGKCSGVAVSSTYC | 112 | 56 | 106 | 265 |
| SGKCSGVAVSSTYCS | 121 | 64 | 65 | 266 |
| GKCSGVAVSSTYCST | 143 | 92 | 103 | 267 |
| KCSGVAVSSTYCSTN | 130 | 88 | 111 | 268 |
| CSGVAVSSTYCSTNH | 165 | 110 | 106 | 269 |
| SGVAVSSTYCSTNHD | 110 | 79 | 84 | 270 |
| GVAVSSTYCSTNHDY | 114 | 79 | 83 | 271 |
| VAVSSTYCSTNHDYT | 114 | 85 | 106 | 272 |
| AVSSTYCSTNHDYTI | 105 | 71 | 102 | 273 |
| VSSTYCSTNHDYTIW | 107 | 78 | 80 | 274 |
| SSTYCSTNHDYTIWM | 107 | 76 | 71 | 275 |
| STYCSTNHDYTIWMP | 99 | 86 | 79 | 276 |
| TYCSTNHDYTIWMPE | 107 | 96 | 87 | 277 |
| YCSTNHDYTIWMPEN | 92 | 47 | 76 | 17 |
| CSTNHDYTIWMPENP | 106 | 52 | 58 | 278 |
| STNHDYTIWMPENPR | 112 | 60 | 77 | 279 |
| TNHDYTIWMPENPRL | 129 | 69 | 91 | 280 |
| NHDYTIWMPENPRLG | 119 | 71 | 108 | 15 |
| HDYTIWMPENPRLGM | 125 | 82 | 110 | 281 |
| DYTIWMPENPRLGMS | 127 | 93 | 106 | 282 |
| YTIWMPENPRLGMSC | 132 | 97 | 111 | 3 |
| TIWMPENPRLGMSCD | 106 | 69 | 93 | 283 |
| IWMPENPRLGMSCDI | 110 | 98 | 87 | 4 |
| WMPENPRLGMSCDIF | 113 | 88 | 97 | 5 |
| MPENPRLGMSCDIFT | 121 | 105 | 107 | 284 |
| PENPRLGMSCDIFTN | 111 | 83 | 94 | 285 |
| ENPRLGMSCDIFTNS | 118 | 71 | 101 | 286 |
| NPRLGMSCDIFTNSR | 113 | 90 | 82 | 287 |
| PRLGMSCDIFTNSRG | 112 | 72 | 108 | 288 |
| RLGMSCDIFTNSRGK | 106 | 88 | 92 | 289 |
| LGMSCDIFTNSRGKR | 110 | 76 | 100 | 290 |
| GMSCDIFTNSRGKRA | 120 | 54 | 71 | 291 |
| MSCDIFTNSRGKRAS | 110 | 46 | 71 | 292 |
| SCDIFTNSRGKRASK | 111 | 44 | 89 | 293 |
| CDIFTNSRGKRASKG | 104 | 42 | 133 | 294 |
| DIFTNSRGKRASKGS | 107 | 70 | 114 | 295 |
| IFTNSRGKRASKGSE | 125 | 77 | 97 | 296 |
| FTNSRGKRASKGSET | 111 | 83 | 90 | 297 |
| TNSRGKRASKGSETC | 108 | 68 | 89 | 298 |
| NSRGKRASKGSETCG | 100 | 92 | 63 | 299 |
| SRGKRASKGSETCGF | 93 | 64 | 70 | 300 |
| RGKRASKGSETCGFV | 104 | 75 | 87 | 301 |
| GKRASKGSETCGFVD | 124 | 92 | 97 | 302 |
| KRASKGSETCGFVDE | 106 | 92 | 97 | 303 |
| RASKGSETCGFVDER | 110 | 86 | 90 | 304 |
| ASKGSETCGFVDERG | 108 | 97 | 106 | 305 |
| SKGSETCGFVDERGL | 102 | 92 | 104 | 306 |
| KGSETCGFVDERGLY | 97 | 90 | 100 | 307 |
| GSETCGFVDERGLYK | 115 | 57 | 56 | 308 |
| SETCGFVDERGLYKS | 116 | 33 | 71 | 309 |
| ETCGFVDERGLYKSL | 120 | 64 | 85 | 310 |
| TCGFVDERGLYKSLK | 120 | 47 | 104 | 311 |
| CGFVDERGLYKSLKG | 115 | 72 | 94 | 312 |
| GFVDERGLYKSLKGA | 120 | 84 | 104 | 313 |
| FVDERGLYKSLKGAC | 121 | 86 | 116 | 314 |
| VDERGLYKSLKGACK | 108 | 50 | 82 | 315 |
| DERGLYKSLKGACKL | 119 | 90 | 76 | 316 |
| ERGLYKSLKGACKLK | 118 | 90 | 101 | 317 |
| RGLYKSLKGACKLKL | 121 | 90 | 107 | 318 |
| GLYKSLKGACKLKLC | 129 | 94 | 91 | 319 |
| LYKSLKGACKLKLCG | 136 | 93 | 94 | 320 |
| YKSLKGACKLKLCGV | 112 | 80 | 79 | 321 |
| KSLKGACKLKLCGVL | 113 | 129 | 91 | 322 |
| SLKGACKLKLCGVLG | 111 | 200 | 99 | 6 |
| LKGACKLKLCGVLGL | 90 | 340 | 100 | 7 |
| KGACKLKLCGVLGLR | 111 | 181 | 50 | 8 |
| GACKLKLCGVLGLRL | 134 | 123 | 64 | 9 |
| ACKLKLCGVLGLRLM | 117 | 148 | 79 | 10 |
| CKLKLCGVLGLRLMD | 111 | 410 | 88 | 11 |
| KLKLCGVLGLRLMDG | 120 | 273 | 101 | 12 |
| LKLCGVLGLRLMDGT | 145 | 918 | 100 | 13 |
| KLCGVLGLRLMDGTW | 132 | 3152 | 96 | 14 |
| LCGVLGLRLMDGTWV | 138 | 83 | 111 | 323 |
| CGVLGLRLMDGTWVA | 117 | 99 | 96 | 324 |
| GVLGLRLMDGTWVAM | 148 | 89 | 107 | 325 |
| VLGLRLMDGTWVAMQ | 141 | 90 | 107 | 326 |
| LGLRLMDGTWVAMQT | 115 | 102 | 113 | 327 |
| GLRLMDGTWVAMQTS | 138 | 104 | 108 | 328 |
| LRLMDGTWVAMQTSN | 114 | 103 | 96 | 329 |
| RLMDGTWVAMQTSNE | 113 | 100 | 99 | 330 |
| LMDGTWVAMQTSNET | 106 | 96 | 102 | 331 |
| MDGTWVAMQTSNETK | 97 | 97 | 85 | 332 |
| DGTWVAMQTSNETKW | 114 | 69 | 63 | 333 |
| GTWVAMQTSNETKWC | 113 | 58 | 61 | 334 |
| TWVAMQTSNETKWCP | 118 | 78 | 100 | 335 |
| WVAMQTSNETKWCPP | 114 | 50 | 111 | 336 |
| VAMQTSNETKWCPPD | 104 | 86 | 97 | 337 |
| AMQTSNETKWCPPDQ | 114 | 104 | 85 | 338 |
| MQTSNETKWCPPDQL | 132 | 104 | 112 | 339 |
| QTSNETKWCPPDQLV | 120 | 92 | 90 | 340 |
| TSNETKWCPPDQLVN | 111 | 97 | 88 | 341 |
| SNETKWCPPDQLVNL | 129 | 99 | 94 | 342 |
| NETKWCPPDQLVNLH | 128 | 90 | 106 | 343 |
| ETKWCPPDQLVNLHD | 120 | 105 | 100 | 344 |
| TKWCPPDQLVNLHDF | 125 | 85 | 97 | 345 |
| KWCPPDQLVNLHDFR | 113 | 89 | 97 | 346 |
| WCPPDQLVNLHDFRS | 119 | 101 | 114 | 347 |
| CPPDQLVNLHDFRSD | 137 | 93 | 115 | 348 |
| PPDQLVNLHDFRSDE | 120 | 107 | 118 | 349 |
| PDQLVNLHDFRSDEI | 106 | 35 | 43 | 350 |
| DQLVNLHDFRSDEIE | 117 | 54 | 88 | 351 |
| QLVNLHDFRSDEIEH | 113 | 60 | 89 | 352 |
| LVNLHDFRSDEIEHL | 104 | 47 | 106 | 353 |
| VNLHDFRSDEIEHLV | 129 | 83 | 103 | 354 |
| NLHDFRSDEIEHLVV | 113 | 83 | 97 | 355 |
| LHDFRSDEIEHLVVE | 115 | 93 | 110 | 356 |
| HDFRSDEIEHLVVEE | 107 | 69 | 78 | 357 |
| DFRSDEIEHLVVEEL | 103 | 99 | 86 | 358 |
| FRSDEIEHLVVEELV | 114 | 86 | 101 | 359 |
| RSDEIEHLVVEELVR | 138 | 100 | 93 | 360 |
| SDEIEHLVVEELVRK | 117 | 101 | 97 | 361 |
| DEIEHLVVEELVRKR | 123 | 94 | 90 | 362 |
| EIEHLVVEELVRKRE | 113 | 82 | 86 | 363 |
| IEHLVVEELVRKREE | 129 | 90 | 100 | 364 |
| EHLVVEELVRKREEC | 114 | 82 | 76 | 365 |
| HLVVEELVRKREECL | 123 | 82 | 111 | 366 |
| LVVEELVRKREECLD | 100 | 64 | 65 | 367 |
| VVEELVRKREECLDA | 108 | 62 | 90 | 368 |
| VEELVRKREECLDAL | 111 | 58 | 84 | 369 |
| EELVRKREECLDALE | 112 | 69 | 118 | 370 |
| ELVRKREECLDALES | 113 | 82 | 97 | 371 |
| LVRKREECLDALESI | 130 | 86 | 107 | 372 |
| VRKREECLDALESIM | 181 | 58 | 111 | 373 |
| RKREECLDALESIMT | 110 | 73 | 96 | 374 |
| KREECLDALESIMTT | 113 | 102 | 83 | 375 |
| REECLDALESIMTTK | 110 | 94 | 94 | 376 |
| EECLDALESIMTTKS | 120 | 82 | 98 | 377 |
| ECLDALESIMTTKSV | 112 | 91 | 103 | 378 |
| CLDALESIMTTKSVS | 146 | 101 | 106 | 379 |
| LDALESIMTTKSVSF | 116 | 97 | 92 | 380 |
| DALESIMTTKSVSFR | 120 | 104 | 105 | 381 |
| ALESIMTTKSVSFRR | 132 | 97 | 107 | 382 |
| LESIMTTKSVSFRRL | 114 | 48 | 94 | 383 |
| ESIMTTKSVSFRRLS | 111 | 62 | 61 | 384 |
| SIMTTKSVSFRRLSH | 130 | 54 | 92 | 385 |
| IMTTKSVSFRRLSHL | 101 | 43 | 85 | 386 |
| MTTKSVSFRRLSHLR | 116 | 59 | 74 | 387 |
| TTKSVSFRRLSHLRK | 118 | 66 | 94 | 388 |
| TKSVSFRRLSHLRKL | 125 | 83 | 103 | 389 |
| KSVSFRRLSHLRKLV | 124 | 108 | 111 | 390 |
| SVSFRRLSHLRKLVP | 123 | 64 | 101 | 391 |
| VSFRRLSHLRKLVPG | 111 | 90 | 55 | 392 |
| SFRRLSHLRKLVPGF | 110 | 92 | 75 | 18 |
| FRRLSHLRKLVPGFG | 108 | 90 | 106 | 393 |
| RRLSHLRKLVPGFGK | 143 | 92 | 85 | 394 |
| RLSHLRKLVPGFGKA | 123 | 93 | 93 | 395 |
| LSHLRKLVPGFGKAY | 139 | 96 | 93 | 396 |
| SHLRKLVPGFGKAYT | 132 | 113 | 118 | 397 |
| HLRKLVPGFGKAYTI | 111 | 99 | 116 | 398 |
| LRKLVPGFGKAYTIF | 118 | 83 | 116 | 399 |
| RKLVPGFGKAYTIFN | 115 | 47 | 48 | 400 |
| KLVPGFGKAYTIFNK | 114 | 47 | 73 | 401 |
| LVPGFGKAYTIFNKT | 112 | 54 | 83 | 402 |
| VPGFGKAYTIFNKTL | 114 | 58 | 96 | 403 |
| PGFGKAYTIFNKTLM | 113 | 78 | 118 | 404 |
| GFGKAYTIFNKTLME | 123 | 78 | 98 | 405 |
| FGKAYTIFNKTLMEA | 151 | 90 | 85 | 406 |
| GKAYTIFNKTLMEAD | 127 | 76 | 100 | 407 |
| KAYTIFNKTLMEADA | 123 | 101 | 76 | 408 |
| AYTIFNKTLMEADAH | 121 | 86 | 98 | 409 |
| YTIFNKTLMEADAHY | 147 | 104 | 90 | 410 |
| TIFNKTLMEADAHYK | 123 | 107 | 100 | 411 |
| IFNKTLMEADAHYKS | 118 | 100 | 87 | 412 |
| FNKTLMEADAHYKSV | 141 | 111 | 86 | 413 |
| NKTLMEADAHYKSVR | 116 | 104 | 94 | 414 |
| KTLMEADAHYKSVRT | 98 | 91 | 102 | 415 |
| TLMEADAHYKSVRTW | 114 | 100 | 111 | 416 |
| LMEADAHYKSVRTWN | 107 | 73 | 46 | 417 |
| MEADAHYKSVRTWNE | 129 | 62 | 78 | 418 |
| EADAHYKSVRTWNEI | 97 | 58 | 79 | 419 |
| ADAHYKSVRTWNEIL | 100 | 56 | 93 | 420 |
| DAHYKSVRTWNEILP | 121 | 59 | 107 | 421 |
| AHYKSVRTWNEILPS | 160 | 112 | 106 | 422 |
| HYKSVRTWNEILPSK | 130 | 80 | 87 | 423 |
| YKSVRTWNEILPSKG | 137 | 66 | 113 | 424 |
| KSVRTWNEILPSKGC | 125 | 115 | 90 | 425 |
| SVRTWNEILPSKGCL | 138 | 106 | 123 | 426 |
| VRTWNEILPSKGCLR | 124 | 90 | 105 | 427 |
| RTWNEILPSKGCLRV | 127 | 120 | 97 | 428 |
| TWNEILPSKGCLRVG | 146 | 97 | 93 | 429 |
| WNEILPSKGCLRVGG | 136 | 102 | 98 | 430 |
| NEILPSKGCLRVGGR | 130 | 104 | 97 | 431 |
| EILPSKGCLRVGGRC | 112 | 104 | 106 | 432 |
| ILPSKGCLRVGGRCH | 113 | 79 | 112 | 433 |
| LPSKGCLRVGGRCHP | 119 | 77 | 58 | 434 |
| PSKGCLRVGGRCHPH | 138 | 69 | 78 | 435 |
| SKGCLRVGGRCHPHV | 121 | 72 | 87 | 436 |
| KGCLRVGGRCHPHVN | 130 | 68 | 108 | 437 |
| GCLRVGGRCHPHVNG | 125 | 85 | 98 | 438 |
| CLRVGGRCHPHVNGV | 132 | 102 | 103 | 439 |
| LRVGGRCHPHVNGVF | 143 | 104 | 104 | 440 |
| RVGGRCHPHVNGVFF | 143 | 86 | 93 | 441 |
| VGGRCHPHVNGVFFN | 136 | 120 | 92 | 442 |
| GGRCHPHVNGVFFNG | 119 | 86 | 110 | 443 |
| GRCHPHVNGVFFNGI | 113 | 117 | 100 | 444 |
| RCHPHVNGVFFNGII | 141 | 98 | 108 | 445 |
| CHPHVNGVFFNGIIL | 150 | 97 | 94 | 446 |
| HPHVNGVFFNGIILG | 138 | 104 | 89 | 447 |
| PHVNGVFFNGIILGP | 173 | 93 | 117 | 448 |
| HVNGVFFNGIILGPD | 123 | 97 | 108 | 449 |
| VNGVFFNGIILGPDG | 116 | 68 | 94 | 450 |
| NGVFFNGIILGPDGN | 117 | 66 | 62 | 451 |
| GVFFNGIILGPDGNV | 116 | 58 | 84 | 452 |
| VFFNGIILGPDGNVL | 132 | 55 | 82 | 453 |
| FFNGIILGPDGNVLI | 143 | 92 | 119 | 454 |
| FNGIILGPDGNVLIP | 139 | 61 | 99 | 455 |
| NGIILGPDGNVLIPE | 146 | 102 | 89 | 456 |
| GIILGPDGNVLIPEM | 132 | 107 | 107 | 457 |
| IILGPDGNVLIPEMQ | 118 | 85 | 80 | 458 |
| ILGPDGNVLIPEMQS | 134 | 125 | 90 | 459 |
| LGPDGNVLIPEMQSS | 134 | 100 | 99 | 460 |
| GPDGNVLIPEMQSSL | 154 | 86 | 91 | 461 |
| PDGNVLIPEMQSSLL | 129 | 87 | 99 | 462 |
| DGNVLIPEMQSSLLQ | 134 | 123 | 93 | 463 |
| GNVLIPEMQSSLLQQ | 120 | 96 | 85 | 464 |
| NVLIPEMQSSLLQQH | 120 | 72 | 92 | 465 |
| VLIPEMQSSLLQQHM | 104 | 92 | 78 | 466 |
| LIPEMQSSLLQQHME | 111 | 89 | 107 | 467 |
| IPEMQSSLLQQHMEL | 128 | 89 | 60 | 468 |
| PEMQSSLLQQHMELL | 133 | 62 | 79 | 469 |
| EMQSSLLQQHMELLE | 129 | 58 | 94 | 470 |
| MQSSLLQQHMELLES | 113 | 65 | 113 | 471 |
| QSSLLQQHMELLESS | 114 | 82 | 98 | 472 |
| SSLLQQHMELLESSV | 128 | 90 | 106 | 473 |
| SLLQQHMELLESSVI | 163 | 124 | 108 | 474 |
| LLQQHMELLESSVIP | 111 | 78 | 80 | 475 |
| LQQHMELLESSVIPL | 134 | 106 | 91 | 476 |
| QQHMELLESSVIPLV | 134 | 103 | 100 | 477 |
| QHMELLESSVIPLVH | 146 | 98 | 87 | 478 |
| HMELLESSVIPLVHP | 129 | 110 | 114 | 479 |
| MELLESSVIPLVHPL | 125 | 90 | 83 | 480 |
| ELLESSVIPLVHPLA | 133 | 90 | 85 | 481 |
| LLESSVIPLVHPLAD | 117 | 72 | 92 | 482 |
| LESSVIPLVHPLADP | 128 | 90 | 110 | 483 |
| ESSVIPLVHPLADPS | 138 | 104 | 121 | 484 |
| SSVIPLVHPLADPST | 104 | 73 | 60 | 485 |
| SVIPLVHPLADPSTV | 137 | 72 | 64 | 486 |
| VIPLVHPLADPSTVF | 141 | 69 | 92 | 487 |
| IPLVHPLADPSTVFK | 156 | 96 | 130 | 488 |
| PLVHPLADPSTVFKD | 112 | 93 | 90 | 489 |
| LVHPLADPSTVFKDG | 174 | 164 | 106 | 490 |
| VHPLADPSTVFKDGD | 138 | 98 | 111 | 491 |
| HPLADPSTVFKDGDE | 141 | 74 | 100 | 492 |
| PLADPSTVFKDGDEA | 125 | 99 | 84 | 493 |
| LADPSTVFKDGDEAE | 116 | 68 | 86 | 494 |
| ADPSTVFKDGDEAED | 152 | 147 | 101 | 495 |
| DPSTVFKDGDEAEDF | 147 | 98 | 132 | 496 |
| PSTVFKDGDEAEDFV | 143 | 104 | 105 | 497 |
| STVFKDGDEAEDFVE | 120 | 104 | 93 | 498 |
| TVFKDGDEAEDFVEV | 124 | 107 | 92 | 499 |
| VFKDGDEAEDFVEVH | 106 | 100 | 125 | 500 |
| FKDGDEAEDFVEVHL | 76 | 65 | 85 | 501 |
| KDGDEAEDFVEVHLP | 93 | 72 | 62 | 502 |
| DGDEAEDFVEVHLPD | 123 | 85 | 97 | 503 |
| GDEAEDFVEVHLPDV | 124 | 46 | 93 | 504 |
| DEAEDFVEVHLPDVH | 136 | 68 | 105 | 505 |
| EAEDFVEVHLPDVHN | 117 | 76 | 97 | 506 |
| AEDFVEVHLPDVHNQ | 138 | 123 | 114 | 507 |
| EDFVEVHLPDVHNQV | 141 | 90 | 114 | 508 |
| DFVEVHLPDVHNQVS | 141 | 96 | 92 | 509 |
| FVEVHLPDVHNQVSG | 143 | 92 | 93 | 510 |
| VEVHLPDVHNQVSGV | 141 | 106 | 117 | 511 |
| EVHLPDVHNQVSGVD | 150 | 91 | 104 | 512 |
| VHLPDVHNQVSGVDL | 114 | 110 | 104 | 513 |
| HLPDVHNQVSGVDLG | 150 | 104 | 96 | 514 |
| LPDVHNQVSGVDLGL | 154 | 104 | 97 | 515 |
| PDVHNQVSGVDLGLP | 129 | 106 | 107 | 516 |
| DVHNQVSGVDLGLPN | 133 | 117 | 124 | 517 |
| VHNQVSGVDLGLPNW | 119 | 100 | 120 | 518 |
| HNQVSGVDLGLPNWG | 106 | 76 | 66 | 519 |
| NQVSGVDLGLPNWGK | 138 | 78 | 103 | 520 |
| Average | 119.5 | 91.9 | 94.1 | |
| StDV | 37.6 | 157.9 | 48.7 | |

TABLE 3

Binding of the human monoclonal antibodies CRJB,
CRJA CR57 to looped/cyclic peptides of the
extracellular domain of glycoprotein G of rabies
virus strain ERA.

| | | | | |
|---|---|---|---|---|
| Amino acid | | | | |
| sequence | CRJB | CR57 | CRJA | SEQ |
| of looped | (10 μg/ | (10 μg/ | (10 μg/ | ID |
| peptide | ml) | ml) | ml) | NO |

| | | | | |
|---|---|---|---|---|
| KFPIYTILDKLGPWS | 64 | 72 | 43 | 114 |
| FPIYTILDKLGPWSP | 63 | 65 | 57 | 115 |
| PIYTILDKLGPWSPI | 77 | 58 | 78 | 116 |
| IYTILDKLGPWSPID | 58 | 66 | 78 | 117 |
| YTILDKLGPWSPIDI | 73 | 75 | 91 | 118 |
| TILDKLGPWSPIDIH | 60 | 85 | 86 | 119 |
| ILDKLGPWSPIDIHH | 46 | 80 | 71 | 120 |
| LDKLGPWSPIDIHHL | 65 | 93 | 82 | 121 |
| DKLGPWSPIDIHHLS | 70 | 104 | 89 | 122 |
| KLGPWSPIDIHHLSC | 65 | 97 | 85 | 123 |
| LGPWSPIDIHHLSCP | 83 | 88 | 72 | 124 |
| GPWSPIDIHHLSCPN | 78 | 78 | 97 | 125 |
| PWSPIDIHHLSCPNN | 75 | 93 | 91 | 126 |
| WSPIDIHHLSCPNNL | 92 | 89 | 151 | 127 |
| SPIDIHHLSCPNNLV | 72 | 94 | 92 | 128 |
| PIDIHHLSCPNNLVV | 70 | 50 | 38 | 129 |
| IDIHHLSCPNNLVVE | 59 | 55 | 55 | 130 |
| DIHHLSCPNNLVVED | 48 | 52 | 62 | 131 |
| IHHLSCPNNLVVEDE | 71 | 46 | 76 | 132 |
| HHLSCPNNLVVEDEG | 58 | 66 | 96 | 133 |
| HLSCPNNLVVEDEGC | 64 | 76 | 92 | 134 |
| LSCPNNLVVEDEGCT | 74 | 72 | 97 | 135 |
| SCPNNLVVEDEGCTN | 69 | 82 | 85 | 136 |
| CPNNLVVEDEGCTNL | 54 | 79 | 84 | 137 |
| PNNLVVEDEGCTNLS | 60 | 100 | 96 | 138 |
| NNLVVEDEGCTNLSG | 75 | 86 | 88 | 139 |
| NLVVEDEGCTNLSGF | 92 | 106 | 74 | 140 |
| LVVEDEGCTNLSGFS | 82 | 76 | 104 | 141 |
| VVEDEGCTNLSGFSY | 66 | 79 | 68 | 142 |
| VEDEGCTNLSGFSYM | 78 | 83 | 86 | 143 |
| EDEGCTNLSGFSYME | 68 | 76 | 54 | 144 |
| DEGCTNLSGFSYMEL | 60 | 1 | 57 | 145 |
| EGCTNLSGFSYMELK | 73 | 39 | 38 | 146 |
| GCTNLSGFSYMELKV | 55 | 63 | 55 | 147 |
| CTNLSGFSYMELKVG | 96 | 70 | 79 | 148 |
| TNLSGFSYMELKVGY | 107 | 39 | 85 | 149 |
| NLSGFSYMELKVGYI | 83 | 68 | 90 | 150 |
| LSGFSYMELKVGYIL | 74 | 72 | 83 | 151 |
| SGFSYMELKVGYILA | 83 | 74 | 69 | 152 |
| GFSYMELKVGYILAI | 57 | 77 | 71 | 153 |
| FSYMELKVGYILAIK | 72 | 104 | 96 | 154 |
| SYMELKVGYILAIKM | 92 | 106 | 96 | 155 |
| YMELKVGYILAIKMN | 83 | 93 | 76 | 156 |
| MELKVGYILAIKMNG | 93 | 71 | 66 | 157 |
| ELKVGYILAIKMNGF | 83 | 84 | 93 | 158 |
| LKVGYILAIKMNGFT | 74 | 58 | 76 | 159 |
| KVGYILAIKMNGFTC | 64 | 96 | 71 | 160 |
| VGYILAIKMNGFTCT | 86 | 97 | 105 | 161 |
| GYILAIKMNGFTCTG | 61 | 87 | 72 | 162 |
| YILAIKMNGFTCTGV | 49 | 55 | 45 | 163 |
| ILAIKMNGFTCTGVV | 72 | 77 | 45 | 164 |
| LAIKMNGFTCTGVVT | 91 | 76 | 79 | 165 |
| AIKMNGFTCTGVVTE | 79 | 69 | 71 | 166 |
| IKMNGFTCTGVVTEA | 86 | 93 | 99 | 167 |
| KMNGFTCTGVVTEAE | 71 | 77 | 83 | 168 |
| MNGFTCTGVVTEAEN | 118 | 85 | 78 | 169 |
| NGFTCTGVVTEAENY | 76 | 92 | 82 | 170 |
| GFTCTGVVTEAENYT | 68 | 94 | 87 | 171 |
| FTCTGVVTEAENYTN | 96 | 123 | 96 | 172 |
| TCTGVVTEAENYTNF | 93 | 107 | 112 | 173 |
| CTGVVTEAENYTNFV | 85 | 92 | 101 | 174 |
| TGVVTEAENYTNFVG | 69 | 92 | 96 | 175 |
| GVVTEAENYTNFVGY | 71 | 83 | 90 | 176 |
| VVTEAENYTNFVGYV | 62 | 80 | 58 | 177 |
| VTEAENYTNFVGYVT | 80 | 84 | 97 | 178 |
| TEAENYTNFVGYVTT | 60 | 75 | 76 | 179 |
| EAENYTNFVGYVTTT | 60 | 55 | 54 | 180 |
| AENYTNFVGYVTTTF | 68 | 58 | 46 | 181 |
| ENYTNFVGYVTTTFK | 80 | 60 | 58 | 182 |
| NYTNFVGYVTTTFKR | 88 | 58 | 85 | 183 |
| YTNFVGYVTTTFKRK | 90 | 71 | 72 | 184 |
| TNFVGYVTTTFKRKH | 99 | 79 | 96 | 185 |
| NFVGYVTTTFKRKHF | 98 | 92 | 83 | 186 |
| FVGYVTTTFKRKHFR | 82 | 117 | 102 | 187 |
| VGYVTTTFKRKHFRP | 85 | 117 | 100 | 188 |
| GYVTTTFKRKHFRPT | 138 | 200 | 101 | 1 |
| YVTTTFKRKHFRPTP | 111 | 146 | 137 | 189 |
| VTTTFKRKHFRPTPD | 83 | 101 | 89 | 190 |
| TTTFKRKHFRPTPDA | 99 | 90 | 93 | 191 |
| TTFKRKHFRPTPDAC | 78 | 86 | 89 | 192 |
| TFKRKHFRPTPDACR | 99 | 112 | 105 | 193 |
| FKRKHFRPTPDACRA | 72 | 148 | 86 | 194 |
| KRKHFRPTPDACRAA | 84 | 94 | 85 | 195 |
| RKHFRPTPDACRAAY | 79 | 72 | 41 | 196 |
| KHFRPTPDACRAAYN | 72 | 70 | 41 | 197 |
| HFRPTPDACRAAYNW | 71 | 65 | 62 | 198 |
| FRPTPDACRAAYNWK | 88 | 90 | 125 | 199 |
| RPTPDACRAAYNWKM | 51 | 76 | 96 | 200 |
| PTPDACRAAYNWKMA | 112 | 114 | 136 | 201 |
| TPDACRAAYNWKMAG | 90 | 125 | 111 | 202 |
| PDACRAAYNWKMAGD | 76 | 97 | 96 | 203 |
| DACRAAYNWKMAGDP | 77 | 133 | 110 | 204 |
| ACRAAYNWKMAGDPR | 93 | 138 | 110 | 205 |
| CRAAYNWKMAGDPRY | 68 | 107 | 111 | 206 |
| RAAYNWKMAGDPRYE | 101 | 141 | 86 | 207 |
| AAYNWKMAGDPRYEE | 90 | 104 | 78 | 208 |
| AYNWKMAGDPRYEES | 77 | 96 | 72 | 209 |
| YNWKMAGDPRYEESL | 89 | 89 | 98 | 210 |
| NWKMAGDPRYEESLH | 78 | 94 | 93 | 211 |
| WKMAGDPRYEESLHN | 77 | 96 | 90 | 212 |
| KMAGDPRYEESLHNP | 45 | 49 | 38 | 213 |
| MAGDPRYEESLHNPY | 62 | 65 | 71 | 214 |
| AGDPRYEESLHNPYP | 54 | 64 | 58 | 215 |
| GDPRYEESLHNPYPD | 82 | 64 | 90 | 216 |
| DPRYEESLHNPYPDY | 65 | 76 | 91 | 217 |
| PRYEESLHNPYPDYR | 79 | 92 | 99 | 218 |
| RYEESLHNPYPDYRW | 71 | 98 | 91 | 219 |
| YEESLHNPYPDYRWL | 50 | 98 | 84 | 220 |
| EESLHNPYPDYRWLR | 85 | 121 | 100 | 221 |
| ESLHNPYPDYRWLRT | 92 | 123 | 106 | 222 |
| SLHNPYPDYRWLRTV | 90 | 104 | 99 | 223 |
| LHNPYPDYRWLRTVK | 93 | 99 | 93 | 224 |
| HNPYPDYRWLRTVKT | 69 | 85 | 65 | 225 |
| NPYPDYRWLRTVKTT | 92 | 89 | 84 | 226 |
| PYPDYRWLRTVKTTK | 92 | 88 | 76 | 227 |
| YPDYRWLRTVKTTKE | 73 | 88 | 92 | 228 |
| PDYRWLRTVKTTKES | 72 | 79 | 90 | 229 |
| DYRWLRTVKTTKESL | 49 | 46 | 45 | 230 |
| YRWLRTVKTTKESLV | 70 | 69 | 58 | 231 |
| RWLRTVKTTKESLVI | 75 | 77 | 71 | 232 |
| WLRTVKTTKESLVII | 78 | 55 | 78 | 233 |
| LRTVKTTKESLVIIS | 68 | 89 | 86 | 234 |
| RTVKTTKESLVIISP | 69 | 88 | 88 | 235 |
| TVKTTKESLVIISPS | 55 | 94 | 92 | 236 |
| VKTTKESLVIISPSV | 92 | 98 | 100 | 237 |
| KTTKESLVIISPSVA | 75 | 111 | 104 | 238 |
| TTKESLVIISPSVAD | 71 | 114 | 108 | 239 |
| TKESLVIISPSVADL | 80 | 99 | 88 | 240 |
| KESLVIISPSVADLD | 85 | 86 | 83 | 241 |
| ESLVIISPSVADLDP | 65 | 99 | 118 | 242 |
| SLVIISPSVADLDPY | 85 | 98 | 87 | 243 |
| LVIISPSVADLDPYD | 102 | 98 | 117 | 244 |
| VIISPSVADLDPYDR | 82 | 90 | 100 | 245 |
| IISPSVADLDPYDRS | 93 | 115 | 106 | 246 |
| ISPSVADLDPYDRSL | 64 | 66 | 46 | 247 |
| SPSVADLDPYDRSLH | 63 | 76 | 51 | 248 |
| PSVADLDPYDRSLHS | 33 | 57 | 62 | 249 |
| SVADLDPYDRSLHSR | 71 | 58 | 83 | 250 |
| VADLDPYDRSLHSRV | 74 | 85 | 89 | 251 |
| ADLDPYDRSLHSRVF | 73 | 93 | 92 | 252 |
| DLDPYDRSLHSRVFP | 68 | 90 | 92 | 253 |
| LDPYDRSLHSRVFPS | 83 | 88 | 98 | 254 |
| DPYDRSLHSRVFPSG | 71 | 106 | 186 | 16 |
| PYDRSLHSRVFPSGK | 90 | 134 | 113 | 255 |
| YDRSLHSRVFPSGKC | 72 | 112 | 86 | 2 |
| DRSLHSRVFPSGKCS | 100 | 91 | 99 | 256 |
| RSLHSRVFPSGKCSG | 93 | 102 | 123 | 257 |
| SLHSRVFPSGKCSGV | 86 | 115 | 97 | 258 |
| LHSRVFPSGKCSGVA | 111 | 110 | 117 | 259 |
| HSRVFPSGKCSGVAV | 104 | 138 | 113 | 260 |
| SRVFPSGKCSGVAVS | 89 | 112 | 92 | 261 |
| RVFPSGKCSGVAVSS | 89 | 75 | 43 | 262 |
| VFPSGKCSGVAVSST | 75 | 79 | 55 | 263 |
| FPSGKCSGVAVSSTY | 74 | 90 | 80 | 264 |
| PSGKCSGVAVSSTYC | 48 | 58 | 73 | 265 |
| SGKCSGVAVSSTYCS | 57 | 77 | 85 | 266 |
| GKCSGVAVSSTYCST | 74 | 79 | 97 | 267 |
| KCSGVAVSSTYCSTN | 83 | 101 | 78 | 268 |
| CSGVAVSSTYCSTNH | 90 | 94 | 94 | 269 |
| SGVAVSSTYCSTNHD | 55 | 79 | 90 | 270 |
| GVAVSSTYCSTNHDY | 80 | 111 | 96 | 271 |
| VAVSSTYCSTNHDYT | 83 | 103 | 88 | 272 |
| AVSSTYCSTNHDYTI | 79 | 129 | 91 | 273 |
| VSSTYCSTNHDYTIW | 61 | 89 | 88 | 274 |
| SSTYCSTNHDYTIWM | 66 | 96 | 90 | 275 |
| STYCSTNHDYTIWMP | 82 | 90 | 90 | 276 |
| TYCSTNHDYTIWMPE | 93 | 104 | 97 | 277 |
| YCSTNHDYTIWMPEN | 71 | 65 | 468 | 17 |
| CSTNHDYTIWMPENP | 72 | 47 | 41 | 278 |
| STNHDYTIWMPENPR | 74 | 72 | 51 | 279 |
| TNHDYTIWMPENPRL | 58 | 40 | 72 | 280 |
| NHDYTIWMPENPRLG | 186 | 170 | 123 | 15 |
| HDYTIWMPENPRLGM | 96 | 88 | 97 | 281 |
| DYTIWMPENPRLGMS | 66 | 83 | 86 | 282 |
| YTIWMPENPRLGMSC | 132 | 191 | 93 | 3 |
| TIWMPENPRLGMSCD | 82 | 97 | 102 | 283 |
| IWMPENPRLGMSCDI | 156 | 329 | 152 | 4 |
| WMPENPRLGMSCDIF | 206 | 199 | 164 | 5 |
| MPENPRLGMSCDIFT | 87 | 107 | 111 | 284 |
| PENPRLGMSCDIFTN | 98 | 116 | 83 | 285 |
| ENPRLGMSCDIFTNS | 88 | 100 | 113 | 286 |
| NPRLGMSCDIFTNSR | 101 | 78 | 91 | 287 |
| PRLGMSCDIFTNSRG | 89 | 87 | 96 | 288 |
| RLGMSCDIFTNSRGK | 104 | 105 | 110 | 289 |
| LGMSCDIFTNSRGKR | 105 | 102 | 104 | 290 |
| GMSCDIFTNSRGKRA | 78 | 79 | 51 | 291 |
| MSCDIFTNSRGKRAS | 73 | 71 | 49 | 292 |
| SCDIFTNSRGKRASK | 79 | 1 | 57 | 293 |
| CDIFTNSRGKRASKG | 90 | 1 | 101 | 294 |
| DIFTNSRGKRASKGS | 82 | 80 | 99 | 295 |
| IFTNSRGKRASKGSE | 75 | 85 | 88 | 296 |
| FTNSRGKRASKGSET | 82 | 89 | 88 | 297 |
| TNSRGKRASKGSETC | 104 | 107 | 104 | 298 |
| NSRGKRASKGSETCG | 60 | 107 | 71 | 299 |
| SRGKRASKGSETCGF | 86 | 96 | 82 | 300 |
| RGKRASKGSETCGFV | 68 | 101 | 102 | 301 |
| GKRASKGSETCGFVD | 71 | 82 | 93 | 302 |
| KRASKGSETCGFVDE | 85 | 120 | 101 | 303 |
| RASKGSETCGFVDER | 90 | 105 | 100 | 304 |
| ASKGSETCGFVDERG | 94 | 96 | 120 | 305 |
| SKGSETCGFVDERGL | 77 | 104 | 99 | 306 |
| KGSETCGFVDERGLY | 72 | 111 | 71 | 307 |
| GSETCGFVDERGLYK | 71 | 64 | 64 | 308 |
| SETCGFVDERGLYKS | 78 | 58 | 56 | 309 |
| ETCGFVDERGLYKSL | 78 | 90 | 75 | 310 |
| TCGFVDERGLYKSLK | 79 | 84 | 100 | 311 |
| CGFVDERGLYKSLKG | 76 | 85 | 90 | 312 |
| GFVDERGLYKSLKGA | 86 | 107 | 87 | 313 |
| FVDERGLYKSLKGAC | 79 | 97 | 92 | 314 |
| VDERGLYKSLKGACK | 80 | 105 | 96 | 315 |
| DERGLYKSLKGACKL | 123 | 152 | 85 | 316 |
| ERGLYKSLKGACKLK | 72 | 100 | 104 | 317 |
| RGLYKSLKGACKLKL | 96 | 96 | 113 | 318 |
| GLYKSLKGACKLKLC | 97 | 86 | 100 | 319 |
| LYKSLKGACKLKLCG | 79 | 91 | 107 | 320 |
| YKSLKGACKLKLCGV | 82 | 96 | 71 | 321 |
| KSLKGACKLKLCGVL | 97 | 106 | 113 | 322 |
| SLKGACKLKLCGVLG | 79 | 129 | 106 | 6 |
| LKGACKLKLCGVLGL | 76 | 105 | 87 | 7 |
| KGACKLKLCGVLGLR | 60 | 78 | 50 | 8 |
| GACKLKLCGVLGLRL | 79 | 73 | 54 | 9 |
| ACKLKLCGVLGLRLM | 92 | 111 | 71 | 10 |
| CKLKLCGVLGLRLMD | 74 | 64 | 91 | 11 |
| KLKLCGVLGLRLMDG | 63 | 13 | 79 | 12 |
| LKLCGVLGLRLMDGT | 72 | 89 | 90 | 13 |
| KLCGVLGLRLMDGTW | 68 | 120 | 82 | 14 |
| LCGVLGLRLMDGTWV | 104 | 128 | 106 | 323 |
| CGVLGLRLMDGTWVA | 91 | 110 | 101 | 324 |
| GVLGLRLMDGTWVAM | 83 | 118 | 104 | 325 |
| VLGLRLMDGTWVAMQ | 106 | 94 | 108 | 326 |
| LGLRLMDGTWVAMQT | 108 | 92 | 97 | 327 |
| GLRLMDGTWVAMQTS | 99 | 120 | 100 | 328 |
| LRLMDGTWVAMQTSN | 72 | 98 | 92 | 329 |
| RLMDGTWVAMQTSNE | 89 | 96 | 82 | 330 |
| LMDGTWVAMQTSNET | 76 | 106 | 92 | 331 |
| MDGTWVAMQTSNETK | 82 | 114 | 90 | 332 |
| DGTWVAMQTSNETKW | 58 | 56 | 45 | 333 |
| GTWVAMQTSNETKWC | 85 | 71 | 62 | 334 |
| TWVAMQTSNETKWCP | 89 | 87 | 84 | 335 |
| WVAMQTSNETKWCPP | 34 | 1 | 100 | 336 |
| VAMQTSNETKWCPPD | 66 | 45 | 90 | 337 |
| AMQTSNETKWCPPDQ | 58 | 84 | 90 | 338 |
| MQTSNETKWCPPDQL | 33 | 138 | 74 | 339 |
| QTSNETKWCPPDQLV | 62 | 118 | 106 | 340 |
| TSNETKWCPPDQLVN | 57 | 134 | 96 | 341 |
| SNETKWCPPDQLVNL | 93 | 129 | 102 | 342 |
| NETKWCPPDQLVNLH | 103 | 111 | 125 | 343 |
| ETKWCPPDQLVNLHD | 77 | 102 | 118 | 344 |
| TKWCPPDQLVNLHDF | 68 | 107 | 113 | 345 |
| KWCPPDQLVNLHDFR | 100 | 118 | 102 | 346 |
| WCPPDQLVNLHDFRS | 106 | 105 | 111 | 347 |
| CPPDQLVNLHDFRSD | 123 | 137 | 92 | 348 |
| PPDQLVNLHDFRSDE | 83 | 101 | 97 | 349 |
| PDQLVNLHDFRSDEI | 73 | 70 | 46 | 350 |
| DQLVNLHDFRSDEIE | 27 | 46 | 63 | 351 |
| QLVNLHDFRSDEIEH | 44 | 47 | 66 | 352 |
| LVNLHDFRSDEIEHL | 23 | 1 | 93 | 353 |
| VNLHDFRSDEIEHLV | 56 | 97 | 84 | 354 |
| NLHDFRSDEIEHLVV | 62 | 90 | 86 | 355 |
| LHDFRSDEIEHLVVE | 65 | 40 | 90 | 356 |
| HDFRSDEIEHLVVEE | 79 | 24 | 111 | 357 |
| DFRSDEIEHLVVEEL | 58 | 127 | 93 | 358 |
| FRSDEIEHLVVEELV | 79 | 132 | 94 | 359 |
| RSDEIEHLVVEELVR | 93 | 136 | 107 | 360 |
| SDEIEHLVVEELVRK | 85 | 96 | 99 | 361 |
| DEIEHLVVEELVRKR | 106 | 113 | 106 | 362 |
| EIEHLVVEELVRKRE | 89 | 107 | 93 | 363 |
| IEHLVVEELVRKREE | 112 | 103 | 112 | 364 |
| EHLVVEELVRKREEC | 83 | 89 | 93 | 365 |
| HLVVEELVRKREECL | 105 | 110 | 110 | 366 |
| LVVEELVRKREECLD | 76 | 68 | 50 | 367 |
| VVEELVRKREECLDA | 5 | 30 | 59 | 368 |
| VEELVRKREECLDAL | 27 | 55 | 69 | 369 |
| EELVRKREECLDALE | 2 | 79 | 104 | 370 |
| ELVRKREECLDALES | 71 | 93 | 98 | 371 |
| LVRKREECLDALESI | 82 | 105 | 101 | 372 |
| VRKREECLDALESIM | 66 | 105 | 101 | 373 |
| RKREECLDALESIMT | 96 | 132 | 129 | 374 |
| KREECLDALESIMTT | 64 | 137 | 100 | 375 |
| REECLDALESIMTTK | 79 | 89 | 92 | 376 |
| EECLDALESIMTTKS | 70 | 105 | 105 | 377 |
| ECLDALESIMTTKSV | 90 | 96 | 110 | 378 |
| CLDALESIMTTKSVS | 90 | 111 | 123 | 379 |
| LDALESIMTTKSVSF | 106 | 108 | 90 | 380 |
| DALESIMTTKSVSFR | 127 | 127 | 110 | 381 |
| ALESIMTTKSVSFRR | 111 | 136 | 108 | 382 |
| LESIMTTKSVSFRRL | 78 | 94 | 91 | 383 |
| ESIMTTKSVSFRRLS | 92 | 80 | 49 | 384 |
| SIMTTKSVSFRRLSH | 25 | 69 | 72 | 385 |
| IMTTKSVSFRRLSHL | 42 | 74 | 63 | 386 |
| MTTKSVSFRRLSHLR | 8 | 68 | 79 | 387 |
| TTKSVSFRRLSHLRK | 72 | 92 | 97 | 388 |
| TKSVSFRRLSHLRKL | 94 | 88 | 91 | 389 |
| KSVSFRRLSHLRKLV | 97 | 114 | 88 | 390 |
| SVSFRRLSHLRKLVP | 84 | 94 | 98 | 391 |
| VSFRRLSHLRKLVPG | 94 | 141 | 99 | 392 |
| SFRRLSHLRKLVPGF | 87 | 143 | 320 | 18 |
| FRRLSHLRKLVPGFG | 54 | 128 | 111 | 393 |
| RRLSHLRKLVPGFGK | 88 | 111 | 96 | 394 |
| RLSHLRKLVPGFGKA | 111 | 111 | 106 | 395 |
| LSHLRKLVPGFGKAY | 123 | 121 | 93 | 396 |
| SHLRKLVPGFGKAYT | 103 | 143 | 160 | 397 |
| HLRKLVPGFGKAYTI | 93 | 118 | 120 | 398 |
| LRKLVPGFGKAYTIF | 105 | 92 | 87 | 399 |
| RKLVPGFGKAYTIFN | 79 | 52 | 44 | 400 |
| KLVPGFGKAYTIFNK | 71 | 54 | 71 | 401 |
| LVPGFGKAYTIFNKT | 58 | 87 | 58 | 402 |
| VPGFGKAYTIFNKTL | 42 | 74 | 87 | 403 |
| PGFGKAYTIFNKTLM | 79 | 110 | 94 | 404 |
| GFGKAYTIFNKTLME | 83 | 94 | 86 | 405 |
| FGKAYTIFNKTLMEA | 78 | 114 | 96 | 406 |
| GKAYTIFNKTLMEAD | 100 | 114 | 107 | 407 |
| KAYTIFNKTLMEADA | 92 | 137 | 104 | 408 |
| AYTIFNKTLMEADAH | 78 | 118 | 97 | 409 |
| YTIFNKTLMEADAHY | 79 | 119 | 108 | 410 |
| TIFNKTLMEADAHYK | 91 | 114 | 96 | 411 |
| IFNKTLMEADAHYKS | 86 | 107 | 98 | 412 |
| FNKTLMEADAHYKSV | 129 | 124 | 101 | 413 |
| NKTLMEADAHYKSVR | 97 | 120 | 98 | 414 |
| KTLMEADAHYKSVRT | 97 | 125 | 92 | 415 |
| TLMEADAHYKSVRTW | 87 | 89 | 89 | 416 |
| LMEADAHYKSVRTWN | 72 | 41 | 43 | 417 |
| MEADAHYKSVRTWNE | 86 | 69 | 68 | 418 |
| EADAHYKSVRTWNEI | 76 | 78 | 63 | 419 |
| ADAHYKSVRTWNEIL | 82 | 69 | 90 | 420 |
| DAHYKSVRTWNEILP | 100 | 90 | 98 | 421 |
| AHYKSVRTWNEILPS | 106 | 106 | 104 | 422 |
| HYKSVRTWNEILPSK | 101 | 112 | 100 | 423 |
| YKSVRTWNEILPSKG | 94 | 117 | 132 | 424 |
| KSVRTWNEILPSKGC | 104 | 148 | 110 | 425 |
| SVRTWNEILPSKGCL | 147 | 151 | 165 | 426 |
| VRTWNEILPSKGCLR | 98 | 121 | 114 | 427 |
| RTWNEILPSKGCLRV | 93 | 107 | 102 | 428 |
| TWNEILPSKGCLRVG | 113 | 132 | 127 | 429 |
| WNEILPSKGCLRVGG | 98 | 112 | 96 | 430 |
| NEILPSKGCLRVGGR | 111 | 104 | 105 | 431 |
| EILPSKGCLRVGGRC | 97 | 132 | 111 | 432 |
| ILPSKGCLRVGGRCH | 91 | 105 | 97 | 433 |
| LPSKGCLRVGGRCHP | 85 | 80 | 52 | 434 |
| PSKGCLRVGGRCHPH | 99 | 92 | 71 | 435 |
| SKGCLRVGGRCHPHV | 87 | 79 | 71 | 436 |
| KGCLRVGGRCHPHVN | 91 | 65 | 102 | 437 |
| GCLRVGGRCHPHVNG | 112 | 103 | 105 | 438 |
| CLRVGGRCHPHVNGV | 104 | 101 | 111 | 439 |
| LRVGGRCHPHVNGVF | 105 | 99 | 96 | 440 |
| RVGGRCHPHVNGVFF | 104 | 107 | 117 | 441 |
| VGGRCHPHVNGVFFN | 64 | 143 | 106 | 442 |
| GGRCHPHVNGVFFNG | 110 | 134 | 107 | 443 |
| GRCHPHVNGVFFNGI | 102 | 110 | 104 | 444 |
| RCHPHVNGVFFNGII | 100 | 104 | 106 | 445 |
| CHPHVNGVFFNGIIL | 101 | 113 | 105 | 446 |
| HPHVNGVFFNGIILG | 99 | 104 | 91 | 447 |
| PHVNGVFFNGIILGP | 134 | 112 | 107 | 448 |
| HVNGVFFNGIILGPD | 92 | 97 | 105 | 449 |
| VNGVFFNGIILGPDG | 96 | 90 | 78 | 450 |
| NGVFFNGIILGPDGN | 85 | 58 | 46 | 451 |
| GVFFNGIILGPDGNV | 85 | 57 | 68 | 452 |
| VFFNGIILGPDGNVL | 93 | 110 | 83 | 453 |
| FFNGIILGPDGNVLI | 96 | 72 | 100 | 454 |
| FNGIILGPDGNVLIP | 88 | 94 | 106 | 455 |
| NGIILGPDGNVLIPE | 85 | 104 | 85 | 456 |
| GIILGPDGNVLIPEM | 93 | 108 | 92 | 457 |
| IILGPDGNVLIPEMQ | 83 | 99 | 107 | 458 |
| ILGPDGNVLIPEMQS | 92 | 143 | 100 | 459 |
| LGPDGNVLIPEMQSS | 94 | 150 | 104 | 460 |
| GPDGNVLIPEMQSSL | 100 | 141 | 112 | 461 |
| PDGNVLIPEMQSSLL | 108 | 110 | 112 | 462 |
| DGNVLIPEMQSSLLQ | 104 | 114 | 107 | 463 |
| GNVLIPEMQSSLLQQ | 103 | 99 | 78 | 464 |
| NVLIPEMQSSLLQQH | 99 | 97 | 110 | 465 |
| VLIPEMQSSLLQQHM | 85 | 114 | 92 | 466 |
| LIPEMQSSLLQQHME | 85 | 98 | 91 | 467 |
| IPEMQSSLLQQHMEL | 83 | 66 | 54 | 468 |
| PEMQSSLLQQHMELL | 82 | 72 | 78 | 469 |
| EMQSSLLQQHMELLE | 98 | 78 | 88 | 470 |
| MQSSLLQQHMELLES | 90 | 72 | 99 | 471 |
| QSSLLQQHMELLESS | 85 | 97 | 99 | 472 |
| SSLLQQHMELLESSV | 76 | 98 | 90 | 473 |
| SLLQQHMELLESSVI | 85 | 113 | 101 | 474 |
| LLQQHMELLESSVIP | 129 | 123 | 165 | 475 |
| LQQHMELLESSVIPL | 93 | 136 | 108 | 476 |
| QQHMELLESSVIPLV | 92 | 141 | 94 | 477 |
| QHMELLESSVIPLVH | 97 | 132 | 111 | 478 |
| HMELLESSVIPLVHP | 104 | 118 | 106 | 479 |
| MELLESSVIPLVHPL | 100 | 115 | 94 | 480 |
| ELLESSVIPLVHPLA | 88 | 112 | 73 | 481 |
| LLESSVIPLVHPLAD | 76 | 93 | 91 | 482 |
| LESSVIPLVHPLADP | 128 | 120 | 114 | 483 |
| ESSVIPLVHPLADPS | 92 | 108 | 91 | 484 |
| SSVIPLVHPLADPST | 80 | 120 | 45 | 485 |
| SVIPLVHPLADPSTV | 106 | 71 | 75 | 486 |
| VIPLVHPLADPSTVF | 92 | 77 | 84 | 487 |
| IPLVHPLADPSTVFK | 107 | 99 | 106 | 488 |
| PLVHPLADPSTVFKD | 90 | 101 | 104 | 489 |
| LVHPLADPSTVFKDG | 116 | 133 | 108 | 490 |
| VHPLADPSTVFKDGD | 79 | 107 | 99 | 491 |
| HPLADPSTVFKDGDE | 93 | 111 | 115 | 492 |
| PLADPSTVFKDGDEA | 97 | 148 | 97 | 493 |
| LADPSTVFKDGDEAE | 90 | 134 | 90 | 494 |
| ADPSTVFKDGDEAED | 72 | 118 | 101 | 495 |
| DPSTVFKDGDEAEDF | 110 | 134 | 110 | 496 |
| PSTVFKDGDEAEDFV | 101 | 118 | 113 | 497 |
| STVFKDGDEAEDFVE | 93 | 106 | 100 | 498 |
| TVFKDGDEAEDFVEV | 90 | 111 | 110 | 499 |
| VFKDGDEAEDFVEVH | 125 | 168 | 104 | 500 |
| FKDGDEAEDFVEVHL | 80 | 106 | 97 | 501 |
| KDGDEAEDFVEVHLP | 71 | 71 | 42 | 502 |
| DGDEAEDFVEVHLPD | 102 | 71 | 71 | 503 |
| GDEAEDFVEVHLPDV | 87 | 87 | 82 | 504 |
| DEAEDFVEVHLPDVH | 104 | 89 | 98 | 505 |
| EAEDFVEVHLPDVHN | 93 | 98 | 105 | 506 |
| AEDFVEVHLPDVHNQ | 90 | 117 | 101 | 507 |
| EDFVEVHLPDVHNQV | 89 | 117 | 104 | 508 |
| DFVEVHLPDVHNQVS | 92 | 113 | 113 | 509 |
| FVEVHLPDVHNQVSG | 101 | 150 | 103 | 510 |
| VEVHLPDVHNQVSGV | 104 | 138 | 120 | 511 |
| EVHLPDVHNQVSGVD | 107 | 125 | 103 | 512 |
| VHLPDVHNQVSGVDL | 94 | 105 | 92 | 513 |
| HLPDVHNQVSGVDLG | 93 | 119 | 87 | 514 |
| LPDVHNQVSGVDLGL | 118 | 116 | 98 | 515 |
| PDVHNQVSGVDLGLP | 104 | 106 | 115 | 516 |
| DVHNQVSGVDLGLPN | 113 | 120 | 99 | 517 |
| VHNQVSGVDLGLPNW | 106 | 125 | 106 | 518 |
| HNQVSGVDLGLPNWG | 100 | 78 | 55 | 519 |
| NQVSGVDLGLPNWGK | 128 | 84 | 79 | 520 |
| Average | 83.6 | 96.0 | 92.0 | |
| StDV | 21.4 | 30.3 | 30.3 | |

TABLE 4
Neutralizing potency of CR57 and CRJB against wild-type and escape
viruses.

| | Potency | Potency | | Potency | Potency |
|---|---|---|---|---|---|
| | CR57 | CRJB | | CR57 | CRJB |
| Virus | (IU/mg) | (IU/mg) | Virus | (IU/mg) | (IU/mg) |
| CVS-11 | 3797 | 605 | CVS-11 | 3797 | 605 |
| E57A2 | 0 | <0.2 | EJB2B | 0.004 | 0.6 |
| E57A3 | 0 | 419 | EJB2C | <0.004 | 2 |
| E57B1 | 0 | 93 | EJB2D | <0.004 | 3 |
| E57B2 | 0 | <0.3 | EJB2E | <0.2 | <0.3 |
| E57B3 | 0 | 419 | EJB2F | <0.06 | 3 |
| E57C3 | 0 | 31 | EJB3F | <0.04 | 0.06 |

TABLE 5
Occurrence of amino acid residues in the minimal binding region
within genotype 1 rabies viruses.

| Wild | | | | | | |
|---|---|---|---|---|---|---|
| type | K | L | C | G | V | L |
| | K (99.6%)* | L | C | G (98.7%) | V | L (70.7%) |
| | | (100%) | (100%) | | (99.6%) | |
| | R (0.4%) | | | E (0.9%) | I (0.4%) | P (26.7%) |
| | | | | R (0.4%) | | S (2.6%) |

*Percentage of occurrence of each amino acid is shown within 229 rabies virus isolates.

### REFERENCES

Dietzschold B. et al. 1990. Structural and immunological characterization of a linear virus-neutralizing epitope of the rabies virus glycoprotein and its possible use in a synthetic vaccine. J. of Virol. 64, 3804-3809.

Lafon M. et al. 1983. Antigenic sites on the CVS rabies virus glycoprotein: analysis with monoclonal antibodies. J. Gen. Virol. 64, 843-851.

Luo T. R. et al. 1997. A virus-neutralizing epitope on the glycoprotein of rabies virus that contains Trp251 is a linear epitope. Virus Research 51, 35-41.

Slootstra J. W. et al. 1996. Structural aspects of antibody-antigen interaction revealed through small random peptide libraries. Mol. Divers. 1, 87-96.

1. A peptide derived from a rabies virus glycoprotein, wherein said peptide consists of 6 to 35 amino acids and comprises a linear epitope comprising the amino acid sequence KX₁CGVX₂ (SEQ ID NO:104), wherein X₁ and X₂ are any amino acid residue.

2. The peptide of claim 1, wherein the peptide is derived from the extracellular domain of the rabies virus glycoprotein.

3. The peptide of claim 1, wherein the peptide binds to a CR57 rabies virus neutralizing antibody.

4. The peptide of claim 1, wherein the peptide is able to elicit at least one rabies virus neutralizing antibody.

5. The peptide of claim 1, wherein X₁ and X₂ are both amino acid residues comprising nonpolar side chains.

6. The peptide of claim 5, wherein X₁ and X₂ are selected from leucine and alanine.

7. The peptide of claim 1, wherein the peptide is linear.

8. A fusion protein or a conjugate comprising the peptide of claim 1.

9. A multimer of peptides, wherein at least one peptide of said multimer is a peptide of claim 1.

10. A nucleic acid molecule encoding the peptide of claim 1.

11. A vector comprising at least one nucleic acid molecule of claim 10.

12. A host comprising at least one vector of claim 11.

13. The host of claim 12, wherein the host is a cell.

14. A vaccine comprising the peptide of claim 1.

15. The vaccine of claim 14, further comprising a pharmaceutically acceptable adjuvant.

16. A rabies virus neutralizing antibody, wherein the rabies virus neutralizing antibody is able to bind to the peptide of claim 1, wherein the rabies virus neutralizing antibody does not comprise a heavy chain comprising the amino acid sequence of SEQ ID NO:33 and a light chain comprising the amino acid sequence of SEQ ID NO:35.

17. The rabies virus neutralizing antibody of claim 16, wherein said rabies virus neutralizing antibody binds to the linear epitope comprising the amino acid sequence KX₁CGVX₂ (SEQ ID NO:104), wherein X₁ and X₂ are any amino acid residue.

18. The rabies virus neutralizing antibody of claim 16, wherein the rabies virus neutralizing antibody is a monoclonal antibody.

19. The rabies virus neutralizing antibody of claim 16, wherein the rabies virus neutralizing antibody is humanized.

20. A pharmaceutical composition comprising the peptide of claim 1, said composition farther comprising a pharmaceutically acceptable excipient or carrier.

21. A medicament comprising the peptide of claim 1.

22. A method of treating a condition in a subject resulting from a rabies virus comprising administering to the subject the peptide of claim 1.

23. A method of producing a rabies virus neutralizing antibody, said method comprising the steps of:
immunizing an animal with the peptide of claim 1 and
isolating a rabies virus neutralizing antibody from the animal.

24. A nucleic acid molecule encoding the fusion protein or conjugate of claim 8.

25. A nucleic acid molecule encoding the multimer of claim 9.

26. A vaccine comprising the fusion protein of claim 8.

27. A vaccine comprising the multimer of claim 9.

28. A vaccine comprising the nucleic acid molecule of claim 10.

29. A pharmaceutical composition comprising the fusion protein of claim 8, said composition further comprising a pharmaceutically acceptable excipient or carrier.

30. A pharmaceutical composition comprising the multimer of claim 9, said composition further comprising a pharmaceutically acceptable excipient or carrier.

31. A pharmaceutical composition comprising the nucleic acid molecule of claim 10, said composition further comprising a pharmaceutically acceptable excipient or carrier.

32. A pharmaceutical composition comprising the vaccine of claim 14, said composition further comprising a pharmaceutically acceptable excipient or carrier.

33. A pharmaceutical composition comprising the rabies virus neutralizing antibody of claim 16, said composition further comprising a pharmaceutically acceptable excipient or carrier.

34. A medicament comprising the fusion protein of claim 8.

35. A medicament comprising the multimer of claim 9.

36. A medicament comprising the nucleic acid molecule of claim 10.

37. A medicament comprising the vaccine of claim 14.

38. A medicament comprising the rabies virus neutralizing antibody of claim 16.

39. A medicament comprising the pharmaceutical composition of claim 20.

40. A method of treating a condition in a subject resulting from a rabies virus comprising administering to the subject the fusion protein of claim 8.

41. A method of treating a condition in a subject resulting from a rabies virus comprising administering to the subject the multimer of claim 9.

42. A method of treating a condition in a subject resulting from a rabies virus comprising administering to the subject the nucleic acid molecule of claim 10.

43. A method of treating a condition in a subject resulting from a rabies virus comprising administering to the subject the vaccine of claim 14.

44. A method of treating a condition in a subject resulting from a rabies virus comprising administering to the subject the rabies virus neutralizing antibody of claim 16.

45. A method of treating a condition in a subject resulting from a rabies virus comprising administering to the subject the pharmaceutical composition of claim 20.

46. A method of producing a rabies virus neutralizing antibody, said method comprising the steps of:
immunizing an animal with the fusion protein of claim 8 and
isolating a rabies virus neutralizing antibody from the animal.

47. A method of producing a rabies virus neutralizing antibody, said method comprising the steps of:
immunizing an animal with the multimer of claim 9 and
isolating a rabies virus neutralizing antibody from the animal.

48. A method of producing a rabies virus neutralizing antibody, said method comprising the steps of:
immunizing an animal with the nucleic acid molecule of claim 10 and
isolating a rabies virus neutralizing antibody from the animal.

49. A method of producing a rabies virus neutralizing antibody, said method comprising the steps of:
immunizing an animal with the vaccine of claim 14 and
isolating a rabies virus neutralizing antibody from the animal.
